(19) United States
(12) Patent Application Publication
(10) Pub. No.: US 2025/0127759 A1
(43) Pub. Date: Apr. 24, 2025

(54) METHODS AND DOSING REGIMENS COMPRISING A CDK2 INHIBITOR FOR THE TREATMENT OF CANCER

(71) Applicant: PFIZER INC., NEW YORK, NY (US)

(72) Inventors: Jerry LI, San Diego, CA (US); Tun Tun LIN, San Diego, CA (US)

(73) Assignee: PFIZER INC., NEW YORK, NY (US)

(21) Appl. No.: 18/715,080

(22) PCT Filed: Dec. 1, 2022
(86) PCT No.: PCT/IB2022/061648

§ 371 Date: May 30, 2024

Related U.S. Application Data
(60) Provisional application No. 63/285,456, filed on Dec. 2, 2021.

(51) Int. Cl. A61K 31/4155; A61K 31/4196; A61K 31/506; A61K 31/519; A61K 31/565; A61P 35/00

(52) U.S. Cl. CPC A61K 31/4155; A61K 31/4196; A61K 31/506; A61K 31/519; A61K 31/565; A61P 35/00

(57) ABSTRACT

The disclosure provides methods of treating cancer in a subject in need thereof comprising administering to the subject a therapeutically effective amount of PF-07104091, as a monotherapy or in combination with an endocrine therapy agent and/or a CDK4/6 inhibitor.

### BACKGROUND

### Field of the Disclosure

The present disclosure relates to the therapeutic treatment of cancer with a cyclin-dependent kinase 2 (CDK2) inhibitor, (1R,3S)-3-[3-({[3-(methoxymethyl)-1-methyl-1H-pyrazol-5-yl]carbonyl}amino)-1H-pyrazol-5-yl]cyclopentyl propan-2-ylcarbamate (also referred to herein as PF-07104091) or a monohydrate thereof, as a monotherapy or in combination with an endocrine therapeutic agent and/or a CDK4/6 inhibitor. This disclosure also relates to associated combination therapies, pharmaceutical compositions, and pharmaceutical uses.

### Description of Related Art

Cyclin-dependent kinases (CDKs) are important cellular enzymes that perform essential functions in regulating eukaryotic cell division and proliferation. CDK inhibitors may be useful for the treatment of proliferative disorders, including cancer.

Overexpression of CDK2 is associated with abnormal regulation of the cell-cycle. The cyclin E/CDK2 complex plays and important role in regulation of the G1/S transition, histone biosynthesis and centrosome duplication. Progressive phosphorylation of retinoblastoma (Rb) by cyclin D/Cdk4/6 and cyclin E/Cdk2 releases the G1 transcription factor, E2F, and promotes S-phase entry. Activation of cyclin A/CDK2 during early S-phase promotes phosphorylation of endogenous substrates that permit DNA replication and inactivation of E2F, for S-phase completion. (Asghar et al. The history and future of targeting cyclin-dependent kinases in cancer therapy, Nat. Rev. Drug. Discov. 2015; 14(2): 130-146).

Cyclin E, the regulatory cyclin for CDK2, is frequently overexpressed in cancer. Cyclin E amplification or overexpression has long been associated with poor outcomes in breast cancer. (Keyomarsi et al., Cyclin E and survival in patients with breast cancer. N Engl J Med. (2002) 347:1566-75). Cyclin E2 (CCNE2) overexpression is associated with endocrine resistance in breast cancer cells and CDK2 inhibition has been reported to restore sensitivity to tamoxifen or CDK4 inhibitors in tamoxifen-resistant and CCNE2 overexpressing cells. (Caldon et al., Cyclin E2 overexpression is associated with endocrine resistance but not insensitivity to CDK2 inhibition in human breast cancer cells. Mol. Cancer Ther. (2012) 11:1488-99; Herrera-Abreu et al., Early Adaptation and Acquired Resistance to CDK4/6 Inhibition in Estrogen Receptor-Positive Breast Cancer, Cancer Res. (2016) 76: 2301-2313). Cyclin E amplification also reportedly contributes to trastuzumab resistance in HER2+ breast cancer. (Scaltriti et al. Cyclin E amplification/overexpression is a mechanism of trastuzumab resistance in HER2+ breast cancer patients, Proc Natl Acad Sci. (2011) 108: 3761-6). Cyclin E overexpression has also been reported to play a role in basal-like and triple negative breast cancer (TNBC), as well as inflammatory breast cancer. (Elsawaf & Sinn, Triple Negative Breast Cancer: Clinical and Histological Correlations, Breast Care (2011) 6:273-278; Alexander et al., Cyclin E overexpression as a biomarker for combination treatment strategies in inflammatory breast cancer, Oncotarget (2017) 8: 14897-14911.)

High CCNE1 mRNA expression was found to be associated with relative resistance to palbociclib in a gene expression analysis of the PALOMA-3 trial, suggesting a role for CDK2 inhibition in reducing or overcoming resistance to CDK4/6 inhibition. (Turner et al., Cyclin E1 Expression and Palbociclib Efficacy in Previously Treated Hormone Receptor-Positive Metastatic Breast Cancer, J. Clin. Oncol. (2019) 37:1169-1178). Amplification or overexpression of cyclin E1 (CCNE1) is also associated with poor outcomes in ovarian, gastric, endometrial and other cancers. (Nakayama et al., Gene amplification CCNE1 is related to poor survival and potential therapeutic target in ovarian cancer, Cancer (2010) 116: 2621-34; Etemadmoghadam et al., Resistance to CDK2 Inhibitors Is Associated with Selection of Polyploid Cells in CCNE1-Amplified Ovarian Cancer, Clin Cancer Res (2013) 19: 5960-71; Au-Yeung et al., Selective Targeting of Cyclin E1-Amplified High-Grade Serous Ovarian Cancer by Cyclin-Dependent Kinase 2 and AKT Inhibition, Clin. Cancer Res. (2017) 23:1862-1874; Ayhan et al., CCNE1 copy-number gain and overexpression identify ovarian clear cell carcinoma with a poor prognosis, Modern Pathology (2017) 30: 297-303; Ooi et al., Gene amplification of CCNE1, CCND1, and CDK6 in gastric cancers detected by multiplex ligation-dependent probe amplification and fluorescence in situ hybridization, Hum Pathol. (2017) 61: 58-67; Noske et al., Detection of CCNE1/URI (19q12) amplification by in situ hybridisation is common in high grade and type II endometrial cancer, Oncotarget (2017) 8: 14794-14805).

CDK4/6 inhibition has emerged as a promising strategy for cancer therapy, especially for the treatment of endocrine resistant breast cancer (BC). (Rani, A., et. al., Endocrine Resistance in Hormone Receptor Positive Breast Cancer—From Mechanism to Therapy. Front Endocrinol (Lausanne) 10:245, 2019). CDK4/6 inhibitors (e.g., palbociclib, abemaciclib, ribociclib) when dosed in combination with endocrine therapy, have significantly improved progression-free survival and/or overall survival for patients with HR-positive/HER2-negative metastatic breast cancer. (Spring, L. M., et. al., Cyclin-dependent kinase 4 and 6 inhibitors for hormone receptor-positive breast cancer: past, present, and future. Lancet, 395, 817-827, 2020).

While CDK4/6 inhibitors have shown significant efficacy in HR-positive metastatic breast cancer, they are associated with dose-limiting hematologic toxicities, primarily neutropenia, as well as gastrointestinal toxicities. As with other kinase inhibitors, the effectiveness of CDK4/6 inhibitors may be limited over time by the development of primary or acquired resistance.

The compound (1R,3S)-3-[3-({[3-(methoxymethyl)-1-methyl-1H-pyrazol-5-yl]carbonyl}amino)-1H-pyrazol-5-yl]cyclopentyl propan-2-ylcarbamate (hereinafter PF-07104091) or a monohydrate thereof is a potent and selective inhibitor of cyclin dependent kinase 2 (CDK2), having the structure of formula (I):

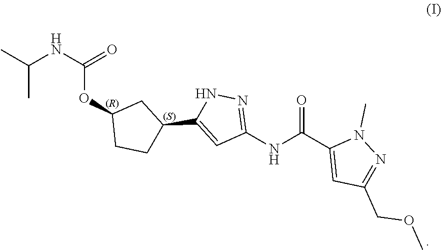

PF-07104091 (Pfizer Inc.) is currently in clinical development for the treatment of certain cancers. Preparation of PF-07104091 is disclosed in International Patent Publication No. WO 2020/157652 and in U.S. Pat. No. 11,014,911, the contents of each which are incorporated herein by reference in their entirety. PF-07104091 also has a name (1R,3S)-3-(3-(3-(methoxymethyl)-1-methyl-1H-pyrazole-5-carboxamido)-1H-pyrazol-5-yl)cyclopentyl isopropylcarbamate, as generated by ChemDraw 20.1.1.

There is a need for appropriate and efficacious dosing regimens of PF-07104091 as a monotherapy and in combination therapies for treating cancer, while maintaining an acceptable safety profile and minimizing adverse events.

### BRIEF SUMMARY

The disclosure relates to both single agent and combination therapies for treating cancer, which comprise the CDK2 inhibitor, PF-07104091, or a pharmaceutically acceptable solvate thereof.

The disclosure also provides a method of treating cancer in a subject in need thereof comprising orally administering to the subject a therapeutically effective amount of PF-07104091. Particularly, the method includes administering to the subject a pharmaceutical composition comprising a therapeutically effective amount of PF-07104091 in a total daily dose of from about 150 mg to about 1000 mg per day, in certain embodiments, from about 75 mg to about 500 mg twice per day (BID).

In certain embodiments, the disclosure provides a method of treating cancer in a subject in need thereof comprising administering to the subject a therapeutically effective amount of PF-07104091 and an endocrine therapy agent. In embodiments, the endocrine therapy agent is an aromatase inhibitor, a selective estrogen receptor degrader (SERD), or a selective estrogen receptor modulator (SERM). In certain embodiments, the endocrine therapy agent is fulvestrant, tamoxifen, toremifene, anastrozole, exemestane, or letrozole.

In certain embodiments, the disclosure also provides methods of treating a cancer in a subject in need thereof comprising administering to the subject a combination therapy which comprises a first therapeutic agent that is PF-07104091, or a pharmaceutically acceptable solvate thereof, a second therapeutic agent that is an EZH2 inhibitor or a pharmaceutically acceptable salt or solvate thereof, and a third therapeutic agent that is fulvestrant.

Accordingly, embodiments herein provide dosing regimens for the use of PF-07104091 as a single agent and in combination therapies for treating cancer, by which therapeutic benefit is obtained while minimizing adverse effects in a subject during the treatment period.

### BRIEF DESCRIPTION OF THE DRAWINGS

FIG. 1 shows the PF-07104091 plasma concentration vs. nominal time on Day 1 following oral dose administration of PF-07104091 as monotherapy.

FIG. 2 shows the PF-07104091 plasma concentration vs. nominal time profiles on Day 15 following repeated BID oral dose administration of PF-07104091 as monotherapy.

### DETAILED DESCRIPTION

The present disclosure may be understood more readily by reference to the following detailed description of the aspects and embodiments of the disclosure and the Examples included herein. It is to be understood that the terminology used herein is for describing specific embodiments only and is not intended to be limiting. It is further to be understood that unless specifically defined herein, the terminology used herein is to be given its traditional meaning as known in the relevant art.

E1. A method of treating cancer in a subject in need thereof comprising administering to the subject an effective amount of PF-07104091, wherein the effective amount is from about 75 mg to about 500 mg twice per day (BID).
E2. The method of embodiment E1, wherein the effective amount is from about 150 mg to about 300 mg BID.
E3. The method of embodiment E1 or E2, wherein the effective amount is about 150 mg BID, about 225 mg BID, or about 300 mg BID.
E4. A method of treating cancer in a subject in need thereof comprising administering to the subject an effective amount of PF-07104091, wherein the effective amount is from about 150 mg to about 1000 mg per day.
E5. The method of embodiment E4, wherein the effective amount is from about 300 mg to about 600 mg per day.
E6. The method of embodiment E4 or E5, wherein the effective amount is about 300 mg per day, about 450 mg per day, or about 600 mg per day.
E7. The method of any one of embodiments E1 to E6, wherein PF-07104091 is administered continuously on a 28 day cycle.
E8. The method of any one of embodiments E1 to E7, wherein PF-07104091 is administered as a tablet or capsule dosage form.
E9. A method of treating cancer in a subject in need thereof comprising administering to the subject an amount of PF-07104091 and an amount of an endocrine therapy agent, wherein the amount of PF-07104091 is from about 75 mg to about 500 mg BID, and the amounts of PF-07104091 and the endocrine therapy agent are together effective to treat cancer.
E10. The method of embodiment E9, wherein the endocrine therapy agent is an aromatase inhibitor, a selective estrogen receptor degrader (SERD), or a selective estrogen receptor modulator (SERM).
E11. The method of embodiment E10, wherein the endocrine therapy agent is selected from the group consisting of letrozole, anastrozole, exemestane, fulvestrant, elacestrant, amcenestrant, giredestrant, RG6171, camizestrant, AZD9496, rintodestrant, ZN-c5, LSZ102, D-0502, LY3484356, SHR9549, tamoxifen, raloxifene, toremifene, lasofoxifene, bazedoxifene and afimoxifene.
E12. The method of any one of embodiments E9 to E11, wherein the endocrine therapy agent is letrozole or fulvestrant.
E13. The method of any one of embodiments E9 to E12, wherein the subject is administered the endocrine therapy agent and PF-07104091 sequentially, concurrently or simultaneously.
E14. The method of any one of embodiments E1 or E13, wherein the subject has been previously treated with chemotherapy, radiotherapy, and/or surgical resection.
E15. The method of any one of embodiments E1 or E13, wherein the subject has been previously treated with a CDK4/6 inhibitor.
E16. The method of any one of embodiments E1 or E13, wherein the subject has been previously treated with an endocrine therapy agent.
E17. The method of any one of embodiments E1 or E16, wherein the subject is a human.
E18. The method of any one of embodiments E1 to E17, wherein the cancer is breast cancer, prostate cancer, lung cancer, liver cancer, kidney cancer, bladder cancer, ovarian cancer, peritoneal cancer, fallopian tube cancer, cervical cancer, uterine cancer, pancreatic cancer, stomach cancer, colorectal cancer, esophageal cancer, head and neck cancer, testicular cancer, adrenal cancer, skin cancer, brain cancer, sarcoma, and lymphoma.
E19. The method of any one of embodiments E1 to E18, wherein the cancer is breast cancer selected from hormone receptor positive (HR+), human epidermal growth factor receptor 2 negative (HER2−) breast cancer and triple negative breast cancer (TNBC).
E20. The method of any one of embodiments E1 to E18, wherein the cancer is lung cancer selected from small cell lung cancer (SCLC) and non-small cell lung cancer (NSCLC).
E21. A method of treating cancer in a subject in need thereof comprising administering to the subject an effective amount of PF-07104091 monohydrate, wherein the effective amount is from about 75 mg to about 500 mg twice per day (BID).
E22. A method of treating cancer in a subject in need thereof comprising administering to the subject an effective amount of PF-07104091 monohydrate, wherein the effective amount is from about 150 mg to about 1000 mg per day.
E23. The method of any one of embodiments E1 to E22, further comprising administering to the subject an effective amount of a CDK4/6 inhibitor.
E24. The method of embodiment E23, wherein the CDK4/6 inhibitor is palbociclib, and wherein the effective amount of palbociclib is about 75 mg once a day (QD), about 100 mg QD, or about 125 mg QD.
E25. The method of embodiment E24, wherein palbociclib is administered on a 28 day cycle with 21 days on treatment followed by 7 days off treatment with palbociclib.
E26. The method of embodiment E23, wherein the CDK4/6 inhibitor is abemaciclib or ribociclib.
E27. The method of any one of embodiments E1 to E22, further comprising administering to the subject an effective amount of a EZH2 inhibitor.
E28. The method of embodiment E27, wherein the EZH2 inhibitor is CPI-1205, GSK126, valemetostat, tazemetostat, PF-06821497, GSK-2816126, 3-deazaneplanocin A, or a pharmaceutically acceptable salt or solvate thereof.
E29. The method of embodiment E28, wherein the EZH2 inhibitor is PF-06821497 having Formula A:

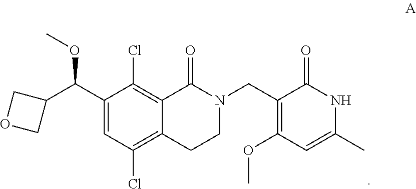 [SMILES: COc1cc(C)[nH]c(=O)c1CN1CCc2c(Cl)cc([C@H](OC)C3COC3)c(Cl)c2C1=O]

### Definitions

As used herein, the singular form “a”, “an”, and “the” include plural references unless indicated otherwise. For example, “a” substituent includes one or more substituents.

The term “about” means having a value falling within an accepted standard of error of the mean, when considered by one of ordinary skill in the art. In some embodiments, the term “about” means within ±10% of the indicated value. For example, a dose of about 150 mg should be understood to mean that the dose may vary between 135 mg and 165 mg.

The disclosure described herein may be suitably practiced in the absence of any element(s) not specifically disclosed herein. Thus, for example, in each instance herein any of the terms “comprising”, “consisting essentially of”, and “consisting of” may be replaced with either of the other two terms.

As used herein, “PF-07104091” may refer to PF-07104091 free base and/or PF-07104091 monohydrate. In preferred embodiments, PF-07104091 as referred to herein is PF-07104091 monohydrate.

As used herein, “dose limiting toxicity” (DLT) refers to the dosage of PF-07104091 that is contraindicative of a further increase in dosage.

As used herein “maximum tolerated dose” (MTD) refers to the highest dosage of PF-07104091 that does not cause unacceptable side effects or intolerable toxicities. MTD is estimated using the mTPI based on observed DLT rate.

As used herein, the term “pharmaceutically acceptable carrier” or “pharmaceutically acceptable excipient” refers to a component that may be included in the compositions described herein, is physiologically suitable for pharmaceutical use, and causes no significant adverse effects to a subject.

As used herein, a “rest period” is the number of days from administration of one complete dose of the active agent to the next administration of one complete dose of the active agent.

As used herein, the term “week” means 7 consecutive days. Thus, a 4-week period is 28 consecutive days starting on any day of the calendar week.

As used herein, the terms “patient” or “subject” refer to any single subject for which therapy is desired or that is participating in a clinical trial, epidemiological study or used as a control, including humans and mammalian veterinary patients, e.g., domestic animals such as cattle, horses, dogs and cats; non-human primates such as monkeys; laboratory animals such as rats, mice, guinea pigs; and captive wild animals such as lions, tigers, and the like. In preferred embodiments, the subject is a human. In some embodiments, the subject is a female. In some embodiments, the subject is a male.

As used herein, an “effective amount” or “therapeutically effective amount” for use and/or for treating a subject refers to an amount that provides, in single or multiple doses, alone, or in combination with one or more other agents, treatments, protocols, or therapeutic regimens, a detectable response of any duration of time (transient, medium or long term), a desired outcome in or an objective or subjective benefit to a subject of any measurable or detectable degree or for any duration of time (e.g., for hours, days, months, years, in remission or cured). Such amounts typically are effective to ameliorate a disease, or one, multiple or all adverse effects/symptoms, consequences or complications of the disease, to a measurable extent, although reducing or inhibiting a progression or worsening of the disease, or providing stability (i.e., not worsening) state of the disease, is considered a satisfactory outcome. The term “therapeutically effective amount” also means an amount of an active agent effective for producing a desired therapeutic effect upon administration to a subject, for example, to stem the growth, or result in the shrinkage, of a cancerous tumor.

The effective amount may vary according to factors such as the disease state, age, sex, and weight of the subject. For prophylactic use, beneficial or desired outcomes may include: eliminating or reducing the risk, lessening the severity, or delaying the onset of the disease. For therapeutic use, beneficial or desired outcomes may include: reducing the incidence or ameliorating one or more symptoms of the disease, reducing the dose of another medication used to treat the disease, enhancing the efficacy or safety of another medication used to treat the disease, delaying the time to disease progression, or prolonging survival.

As used herein, a “treatment cycle” refers to a period of time comprising administration of one or more agents (e.g., PF-07104091, palbociclib, or an endocrine therapy), with or without rest periods between treatment cycles. A treatment cycle may be continuous, i.e., with no rest period between the treatment cycles. Alternatively, a treatment cycle may be intermittent and include a rest period (i.e., a period of dose interruption of one or more days or weeks off treatment) between treatment cycles. In such instances, administration of another agent during the rest period should not interfere or be detrimental to administration of the agent(s) described herein.

For example, a 21 day or 28 day treatment cycle, with 14 or 21 days on treatment, respectively, followed by a 7 day rest period (i.e., treatment interruption) is an example of an intermittent treatment cycle. Treatment cycles with 2 or 3 weeks on treatment and 1 week off treatment are sometimes referred to as a 2/1-week or 3/1-week treatment cycles, respectively. Alternatively, an intermittent treatment cycle may comprise a 7 day cycle, with 5 days on treatment and 2 days off treatment.

As used herein, the term “ameliorate” refers to any reduction in the extent, severity, frequency, and/or likelihood of a symptom or clinical sign characteristic of a particular disease. “Symptom” refers to any subjective evidence of disease or of a subject's condition.

As used herein, “treat” or “treating” a cancer and/or a cancer-associated disease means to administer a mono- or combination therapy according to the present disclosure to a subject, patient or individual having a cancer, or diagnosed with a cancer, to achieve at least one positive therapeutic effect, such as, for example, reduced number of cancer cells, reduced tumor size, reduced rate of cancer cell infiltration into peripheral organs, or reduced rate of tumor metastasis or tumor growth, reversing, alleviating, inhibiting the progress of, or preventing the disorder or condition to which such term applies, or one or more symptoms of such disorder or condition. The term “treatment”, as used herein, unless otherwise indicated, refers to the act of treating as “treating” is defined immediately above. The term “treating” also includes adjuvant and neo-adjuvant treatment of a subject. For the purposes of this disclosure, beneficial or desired clinical results include, but are not limited to, one or more of the following: reducing the proliferation of (or destroying) neoplastic or cancerous cell; inhibiting metastasis or neoplastic cells; shrinking or decreasing the size of tumor; remission of the cancer; decreasing symptoms resulting from the cancer; increasing the quality of life of those suffering from the cancer; decreasing the dose of other medications required to treat the cancer; delaying the progression the cancer; curing the cancer; overcoming one or more resistance mechanisms of the cancer; and/or prolonging survival of patients the cancer. Positive therapeutic effects in cancer can be measured in a number of ways (see, for example, W. A. Weber, J. Nucl. Med. 50:1S-10S (2009)).

The retinoblastoma susceptibility gene (RB1) was the first tumor suppressor gene to be molecularly defined. The retinoblastoma gene product, RB, is frequently mutated or deleted in retinoblastoma and osteosarcoma, and is mutated or deleted with variable frequency in other tumor types, such as prostate cancer (including neuroendocrine prostate carcinoma), breast cancer (including triple negative breast cancer, TNBC), lung cancer (including small cell lung cancer, SCLC, and non-small cell lung cancer, NSCLC), liver cancer, bladder cancer, ovarian cancer, uterine cancer, cervical cancer, stomach cancer, esophageal cancer, head and neck cancer, glioblastoma, and lymphoma. In human cancers, the function of RB may be disrupted through neutralization by a binding protein, (e.g., the human papilloma virus-E7 protein in cervical carcinoma; Ishiji, T, 2000[0021], J Dermatol., 27: 73-86) or deregulation of pathways ultimately responsible for its phosphorylation.

By “RB pathway” it is meant the entire pathway of molecular signaling that includes retinoblastoma protein (RB), and other protein/protein families in the pathway, including but not limited to CDK, E2f, atypical protein kinase C, and Skp2. Inactivation of the RB pathway often results from perturbation of p161NK4a, Cyclin D1, and CDK4. The terms “RB+,” “RB plus,” “RB-proficient” or “RB-positive” may be used to describe cells expressing detectable amounts of functional RB protein. RB-positive includes wild-type and non-mutated RB protein. A wild-type RB (RB-WT) is generally understood to mean that form of the RB protein which is normally present in a corresponding population and which has the function which is currently assigned to this protein. RB-positive may be cells which contain a functional RB gene. Cells which are RB-positive may also be cells that can encode a detectable RB protein function.

The terms “RB−,” “RB minus,” “RB-deficient” or “RB-negative” describe several types of cell where the function of RB is disrupted, including cells which produce no detectable amounts of functional RB protein. Cells that are RB-negative may be cells which do not contain a functional RB gene. Cells that are RB-negative may also be cells that can encode an RB protein, but in which the protein does not function properly. In some embodiments of each of the methods and uses described herein, the cancer is characterized as retinoblastoma wild type (RB-WT). In some embodiments of each of the methods and uses described herein, the cancer is characterized as RB-positive or RB-proficient. Such RB-positive or RB-proficient cancers contain at least some functional retinoblastoma genes. In some embodiments, such RB-WT, RB-positive or RB-proficient cancers are characterized as RB1-WT, RB1-positive or RB1-proficient cancers.

In some embodiments of each of the methods and uses described herein, the cancer is characterized as RB-negative or RB-deficient. Such RB-negative or RB-deficient cancers may be characterized by loss of function mutations, which may encode missense mutations (i.e., encode the wrong amino acid) or nonsense mutations (i.e., encode a stop codon). Alternatively, such RB-negative cancers may be characterized by deletion of all or part of the retinoblastoma gene. In some embodiments, such RB-negative or RB-deficient cancers are characterized as RB1-negative or RB1-deficient.

“Tumor” as it applies to a subject diagnosed with, or suspected of having, a cancer refers to a malignant or potentially malignant neoplasm or tissue mass of any size and includes primary tumors and secondary neoplasms. A solid tumor is an abnormal growth or mass of tissue that usually does not contain cysts or liquid areas. Examples of solid tumors are sarcomas, carcinomas, and lymphomas. Leukemia's (cancers of the blood) generally do not form solid tumors (National Cancer Institute, Dictionary of Cancer Terms).

As used herein, the terms “combination” or “combination therapy” refer to the administration of two or more therapeutic agents of the combination therapy, either alone or in the form of a pharmaceutical composition or medicament. The combination therapy may be administered sequentially, concurrently or simultaneously.

When administering a combination therapy of two or more agents, the agents may be administered on the same treatment cycle or using different cycles. In preferred embodiments, PF-07104091 is administered continuously on a 28 day cycle. Similarly, letrozole is typically administered continuously on a 28 day treatment cycle. Palbociclib is typically administered using an intermittent 28 day cycle, comprising administration of the drug for 21 days, with a rest period of 7 days between the cycles. Fulvestrant is typically administered intramuscularly on days 1, 15, 29 of the first treatment cycle and once monthly thereafter.

Each therapeutic agent of the methods and combination therapies described herein may be administered either alone, or in a medicament (also referred to herein as a pharmaceutical composition) which comprises the therapeutic agent and one or more pharmaceutically acceptable carriers, excipients, or diluents, according to pharmaceutical practice.

The term “sequential” or “sequentially” refers to the administration of each therapeutic agent of the combination therapy, either alone or in a medicament, one after the other, wherein each therapeutic agent can be administered in any order. Sequential administration may be particularly useful when the therapeutic agents in the combination therapy are in different dosage forms, for example, one agent is a tablet and another agent is a sterile liquid, and/or the agents are administered according to different dosing schedules, for example, one agent is administered daily, and the second agent is administered less frequently such as weekly.

The term “concurrently” refers to the administration of each therapeutic agent in a combination therapy, either alone or in separate medicaments, wherein the second therapeutic agent is administered immediately after the first therapeutic agent, but that the therapeutic agents can be administered in any order. In a preferred embodiment the therapeutic agents are administered concurrently.

The term “simultaneous” refers to the administration of each therapeutic agent of the combination therapy in the same medicament, for example as a fixed dose combination comprising two or more drugs in a single dosage form.

A “dosing regimen” refers to the period of administration of one or more drugs, compounds or compositions, comprising one or more treatment cycles, wherein each treatment cycle may include administration of one or more agents at different times, frequencies or amounts, using the same or different routes of administration. Repetition of the administration or dosing regimens, or adjustment of the administration or dosing regimen may be conducted as necessary to achieve the desired treatment effect.

“BID” or “bid” refers to administration of a drug, compound or composition twice a day.

“QD” or “qd” refers to administration of a drug, compound or composition once a day.

“TID” or “tid” refers to administration of a drug, compound or composition three times a day.

PF-07104091 is a selective CDK2 inhibitor of cyclin dependent kinase 2 (CDK2), having the structure of formula (I):

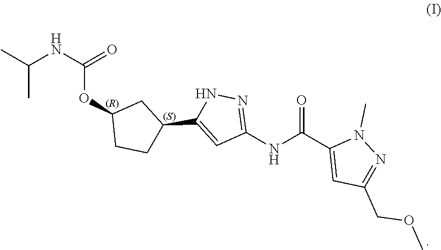

It is understood that the above structural formula of PF-07104091 includes all tautomeric forms which may co-exist and be directly interconverted under the appropriate conditions. For example, in some embodiments, PF-07104091 has a structure of formula (Ia):

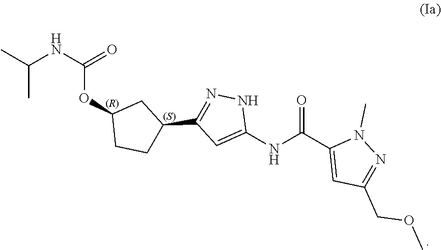

In preferred embodiments of each of the methods, combinations and uses described herein, PF-07104091 is PF-07104091 monohydrate.

PF-07104091 may be administered as a pharmaceutical composition which includes PF-07104091 or a monohydrate thereof as the active pharmaceutical ingredient and a pharmaceutically acceptable carrier. Pharmaceutical compositions may be administered in unit dosage forms, such as tablets or capsules. The amount of PF-07104091 in the unit dosage form typically ranges from about 25 mg to about 250 mg, for example, about 25 mg, about 50 mg, about 75 mg, about 100 mg, about 125 mg, about 150 mg, and so forth.

In certain embodiments, the effective amount of PF-07104091 on a daily basis (i.e., total daily dose) is from about 100 mg to about 1000 mg per day, from about 150 mg to about 1000 mg per day, from about 150 mg to about 900 mg per day, from about 150 mg to about 750 mg per day, from about 150 mg to about 600 mg per day, from about 150 mg to about 450 mg per day, from about 150 mg to about 300 mg per day, from about 300 mg to about 1000 mg per day, from about 300 mg to about 900 mg per day, from about 300 mg to about 750 mg per day, from about 300 mg to about 600 mg per day, from about 300 mg to about 450 mg per day, from about 450 mg to about 1000 mg per day, from about 450 mg to about 900 mg per day, from about 450 mg to about 750 mg per day, from about 450 mg to about 600 mg per day, from about 600 mg to about 1000 mg per day, from about 600 mg to about 900 mg per day, from about 600 mg to about 750 mg per day, from about 750 mg to about 1000 mg per day, or from about 750 mg to about 900 mg per day. In some embodiments, PF-07104091 is administered in a dose of about 300 mg per day, about 350 mg per day, about 400 mg per day, about 450 mg per day, about 500 mg per day, about 550 mg per day, about 600 mg per day, about 650 mg per day, or about 700 mg per day. In preferred embodiments, the effective amount of PF-07104091 is about 300 mg per day, about 450 mg per day, or about 600 mg per day.

In preferred embodiments, PF-07104091 is administered on a twice a day (BID) dosing schedule.

In some embodiments, PF-07104091 is administered in doses of from about 75 mg to about 500 mg BID, from about 75 mg to about 375 mg BID, from about 75 mg to about 300 mg BID, from about 75 mg to about 225 mg BID, from about 75 mg to about 150 mg BID, from about 150 mg to about 500 mg BID, from about 150 mg to about 375 mg BID, from about 150 mg to about 300 mg BID, from about 150 mg to about 225 mg BID, from about 225 mg to about 500 mg BID, from about 225 mg to about 375 mg BID, from about 225 mg to about 300 mg BID, from about 300 mg to about 500 mg BID, from about 300 mg to about 375 mg BID, or from about 375 mg to about 500 mg BID. In some embodiments, PF-07104091 is administered in doses of about 150 mg BID, about 175 mg BID, about 200 mg BID, about 225 mg BID, about 250 mg BID, about 275 mg BID, about 300 mg BID, about 325 mg BID, or about 350 mg BID. In preferred embodiments, PF-07104091 is administered in a dose of from about 150 mg to about 300 mg BID.

In some embodiments, PF-07104091 is administered in doses of about 100 mg BID, about 125 mg BID, about 150 mg BID, about 175 mg BID, about 200 mg BID, about 225 mg BID, about 250 mg BID, about 275 mg BID, about 300 mg BID, about 325 mg BID, about 350 mg BID, or about 375 mg BID. In preferred embodiments, PF-07104091 is administered in a dose of about 150 mg BID, about 225 mg BID, or about 300 mg BID.

In some embodiments, PF-07104091 is administered on a once a day (QD) dosing schedule. In some embodiments, PF-07104091 is administered in doses of from about 150 mg to about 1000 mg QD, from about 150 mg to about 750 mg QD, from about 150 mg to about 600 mg QD, from about 150 mg to about 450 mg QD, from about 150 mg to about 300 mg QD, from about 300 mg to about 1000 mg QD, from about 300 mg to about 750 mg QD, from about 300 mg to about 600 mg QD, from about 300 mg to about 450 mg QD, from about 450 mg to about 1000 mg QD, from about 450 mg to about 750 mg QD, from about 450 mg to about 600 mg QD, from about 600 mg to about 1000 mg QD, from about 600 mg to about 750 mg QD, or from about 750 mg to about 1000 mg QD. In some embodiments, PF-07104091 is administered in a dose of about 300 mg QD, about 350 mg QD, about 400 mg QD, about 450 mg QD, about 500 mg QD, about 550 mg QD, about 600 mg QD, about 650 mg QD, or about 700 mg QD.

In some embodiments, PF-07104091 is administered in doses of about 200 mg QD, about 250 mg QD, about 300 mg QD, about 350 mg QD, about 400 mg QD, about 450 mg QD, about 500 mg QD, about 550 mg QD, about 600 mg QD, about 650 mg QD, about 700 mg QD, or about 750 mg QD, In some such embodiments, PF-07104091 is administered in a dose of about 300 mg QD, about 450 mg QD, or about 600 mg QD.

In some embodiments, the subject is administered PF-07104091 at a dose of any of the therapeutically effective amounts disclosed herein.

The amount of PF-07104091 administered may be increased or decreased based on the weight, age, health, sex, or medical condition of the subject. One of skill in the art would be able to determine the proper dose for a subject based on this disclosure.

PF-07104091 may be administered in treatment cycles, with or without rest periods in between the treatment cycles. A treatment cycle may have a duration of about 7 days, about 14 days, about 21 days, about 28 days, about 35 days and so on, or any days in between. A rest period can be one day or a few days (e.g., 1 day, 2 days, 3 days, 4 days, 5 days, 6 days, and so on), one week, several weeks (e.g., 2 weeks, 3 weeks and so on), or any days in between (e.g., 1 week and 3 days).

In some embodiments, PF-07104091 is administered continuously without a rest period between treatment cycles (i.e., continuous treatment until termination). In some embodiments, PF-07104091 is administered for a treatment cycle (e.g., about 28 days) with or without a rest period. In some embodiments, PF-07104091 is administered for about 28 days with a rest period of about one week. PF-07104091 may be administered for at least about 7 days, about 14 days, about 21 days, about 28 days, about 2 months, about 3 months, about 12 months, about 24 months, and more. In a preferred embodiment, PF-07104091 is administered continuously on a 28 day treatment cycle, without a rest period.

Pharmaceutical compositions comprising PF-07104091 as described herein may be administered on a BID or QD dosing schedule. Pharmaceutical compositions comprising PF-07104091 may be administered with or without food.

Pharmaceutical compositions comprising PF-07104091 may be administered by one or more routes as considered appropriate by a skilled person in the art and depending on the dosage form. The pharmaceutical compositions may be administered with or without food. Formulation of drugs is discussed in Remington's Pharmaceutical Sciences, 18th Ed., (1995) Mack Publishing Co., Easton, Pa. Other examples of drug formulations can be found in Liberman, H. A. and Lachman, L., Eds., Pharmaceutical Dosage Forms, Marcel Decker, Vol 3, 2nd Ed., New York, N.Y. Where the compound is administered orally, it may be formulated as a pill, capsule, tablet, etc. with a pharmaceutically acceptable carrier, glidant, or excipient.

The pharmaceutical compositions may be in one or more dosage forms (e.g., capsule, tablet, powder or liquid). In preferred embodiments, Pharmaceutical compositions comprising PF-07104091 are in tablet or capsule dosage form.

In some embodiments, the pharmaceutical compositions may be administered in an immediate release formulation.

By “immediate release” or “IR” is meant broadly an oral dosage form formulated to release an API immediately after oral administration. In IR formulations, no deliberate effort is made to modify the drug release rate.

### Therapeutic Methods and Uses

In certain embodiments, the disclosure provides a method for treating cancer of a subject in need thereof, which includes administering to the subject a therapeutically effective amount of PF-07104091 as described herein.

In certain embodiments, the disclosure also provides a method for treating cancer of a subject which includes administering to the subject a therapeutically effective amount of PF-07104091 as described herein and an endocrine therapy agent.

In certain embodiments, the disclosure provides a use of PF-07104091 in the manufacture of a medicament for use in the treatment of cancer in a subject in need thereof wherein the medicament is administered in a therapeutically effective unit dosage of PF-07104091 as described herein.

In certain embodiments, the disclosure provides a use of PF-07104091 together with endocrine therapy in the manufacture of a medicament for use in the treatment of cancer in a subject in need thereof wherein the medicament is administered in a therapeutically effective unit dosage of PF-07104091 as described herein.

In certain embodiments, the disclosure provides a medicament comprising a therapeutically effective amount of PF-07104091 as described herein for use in treating cancer in a subject in need thereof.

In embodiments, the disclosure provides a medicament comprising a therapeutically effective amount of PF-07104091 as described herein and an endocrine therapy agent for use in treating cancer in a subject in need thereof.

In certain embodiments of each of the methods, medicaments, combinations and uses described herein, PF-07104091 is administered continuously (i.e., daily).

In certain embodiments, the methods disclosed herein include administering PF-07104091 to a subject having a cancer that is: (1) characterized by amplification or overexpression of CDK2; (2) characterized by amplification or overexpression of CCNE1 and/or CCNE2; (3) characterized by loss or Rb; or (4) resistant to, or having progressed on prior treatment with endocrine therapy, anti-HER2 targeted agents, CDK4/6 inhibition or chemotherapy (e.g., antineoplastic chemotherapeutic agents such as platinum agents, taxanes, anthracyclines or anti-metabolites).

In some embodiments of the methods and uses described herein, the cancer is selected from the group consisting of breast cancer, prostate cancer, lung cancer (including non-small cell lung cancer, NSCLC, and small cell lung cancer, SCLC), liver cancer (including hepatocellular carcinoma, HCC), kidney cancer (including renal cell carcinoma, RCC), bladder cancer (including urothelial carcinomas, such as upper urinary tract urothelial carcinoma, UUTUC), ovarian cancer (including epithelial ovarian cancer, EOC), peritoneal cancer (including primary peritoneal cancer, PPC), fallopian tube cancer, cervical cancer, uterine cancer (including endometrial cancer), pancreatic cancer, stomach cancer, colorectal cancer, esophageal cancer, head and neck cancer (including squamous cell carcinoma of the head and neck (SCCHN), thyroid cancer, and salivary gland cancer), testicular cancer, adrenal cancer, skin cancer (including basal cell carcinoma and melanoma), brain cancer (including astrocytoma, meningioma, and glioblastoma), sarcoma (including osteosarcoma and liposarcoma), and lymphoma (including mantle cell lymphoma, MCL).

In some embodiments of the methods and uses described herein, the cancer is SCLC. In some such embodiments, the SCLC is Rb-negative or Rb-deficient. In some embodiments of the methods and uses described herein, the cancer is NSCLC. In some such embodiments, the NSCLC is characterized by amplification or overexpression of cyclin E1 (CCNE1) and/or cyclin E2 (CCNE2). In some embodiments, the NSCLC is characterized by amplification or overexpression of cyclin E1 (CCNE1). In some such embodiments, the NSCLC is advanced or metastatic NSCLC. In some such embodiments, the NSCLC is advanced or metastatic NSCLC characterized by amplification or overexpression of cyclin E1 (CCNE1). In other embodiments, the NSCLC is lung squamous cell carcinoma (LUSC) or lung adenocarcinoma (LUAD). In a preferred embodiment, the lung cancer is a LUAD. Single-gene drive oncogene drivers of lung adenocarcinomas include, but are not limited to, EGFR, BRAF, and KRAS. About 25% of lung adenocarcinomas are KRAS driven, which may include KRAS G12C and non-G12C driven segments, such as G12A, G12D, G12V, G13D and L19F driven tumors.

In some embodiments, the cancer is lung cancer, including SCLC or NSCLC, and the methods, combinations and uses described herein further comprise an additional anti-cancer agent.

In some embodiments of the methods and uses described herein, the cancer is ovarian cancer (including epithelial ovarian cancer, EOC), peritoneal cancer (including primary peritoneal cancer, PPC), or fallopian tube cancer. In some such embodiments, the cancer is characterized by amplification or overexpression of CCNE1 and/or CCNE2.

In some embodiments of the methods and uses described herein, the cancer is TNBC. In some such embodiments, the TNBC is refractory to CDK4/6 inhibitors, such as palbociclib.

In some embodiments of the methods and uses described herein, the cancer is HR-positive, HER2-negative breast cancer, including advanced or metastatic breast cancer. In some such embodiments, the breast cancer is refractory to CDK4/6 inhibitors, such as palbociclib.

In some embodiments of the methods and uses described herein, the cancer is advanced or metastatic cancer. In some embodiments of the methods and uses described herein, the cancer is early stage or non-metastatic cancer.

In other embodiments, the cancer is breast cancer, including, e.g., ER-positive/HR-positive, HER2-negative breast cancer; ER-positive/HR-positive, HER2-positive breast cancer; triple negative breast cancer (TNBC); or inflammatory breast cancer. In some embodiments, the breast cancer demonstrates primary or acquired resistance to endocrine therapy, anti-HER2 targeted agents, CDK4/6 inhibition, or chemotherapy (e.g., antineoplastic chemotherapeutic agents such as platinum agents, taxanes, anthracyclines or anti-metabolites).

In some embodiments, the breast cancer is advanced or metastatic breast cancer. In some embodiments of each of the foregoing, the breast cancer is characterized by amplification or overexpression of CCNE1 and/or CCNE2.

In some embodiments of the methods provided herein, the abnormal cell growth is cancer characterized by amplification or overexpression of CCNE1 and/or CCNE2. In some embodiments of the methods provided herein, the subject is identified as having a cancer characterized by amplification or overexpression of CCNE1 and/or CCNE2.

In some embodiments, the cancer is breast cancer or ovarian cancer. In some such embodiments, the cancer is breast cancer or ovarian cancer characterized by amplification or overexpression of CCNE1 and/or CCNE2. In some such embodiments, the cancer is (a) breast cancer or ovarian cancer; (b) characterized by amplification or overexpression of CCNE1 or CCNE2; or (c) both (a) and (b).

In some embodiments, the compound of the invention is administered as first line therapy. In other embodiments, the compound of the invention is administered as second (or later) line therapy.

In some embodiments, the compound of the invention is administered as second (or later) line therapy following treatment with an endocrine therapy and/or a CDK4/6 inhibitor. In some embodiments, the compound of the invention is administered as second (or later) line therapy following treatment with an endocrine therapy, e.g., an aromatase inhibitor, a SERM or a SERD. In some embodiments, the compound of the invention is administered as second (or later) line therapy following treatment with a CDK4/6 inhibitor (e.g., palbociclib, ribociclib or abemaciclib, or a pharmaceutically acceptable salt thereof). In some embodiments, the compound of the invention is administered as second (or later) line therapy following treatment with one or more chemotherapy regimens (e.g., including antineoplastic chemotherapeutic agents such as platinum agents, taxanes, anthracyclines or anti-metabolites). In some embodiments, the compound of the invention is administered as second (or later) line therapy following treatment with anti-HER2 targeted agents (e.g., trastuzumab, pertuzumab, lapatinib, or ado-trastuzumab emtansine (T-DM1)).

In certain embodiments, the method disclosed herein further includes administering to a subject a therapeutically effective amount of PF-07104091 and an endocrine therapy agent. An “endocrine therapy agent” may be a biological (large molecule) or chemical (small molecule) compound useful in the treatment of cancer, regardless of mechanism of action. In preferred embodiments, the endocrine therapy agent is an anti-estrogen.

In some embodiments, the endocrine therapy agent is an aromatase inhibitor, a selective estrogen receptor degrader (SERD), or a selective estrogen receptor modulator (SERM). In some embodiments, the endocrine therapy agent is an aromatase inhibitor. In some such embodiments, the aromatase inhibitor is selected from the group consisting of letrozole, anastrozole and exemestane. In one embodiment, the aromatase inhibitor is letrozole. In some embodiments, the endocrine therapy agent is a SERD. In some such embodiments, the SERD is selected from the group consisting of fulvestrant, elacestrant (RAD-1901, Radius Health/Menarini), amcenestrant (SAR439859, Sanofi), giredestrant (GDC9545, Roche), RG6171 (Roche), camizestrant (AZD9833, AstraZeneca), AZD9496 (AstraZeneca), rintodestrant (G1 Therapeutics), ZN-c5 (Zentalis), LSZ102 (Novartis), D-0502 (Inventisbio), LY3484356 (Eli Lilly), and SHR9549 (Jiansu Hengrui Medicine). In some embodiments, the SERD is fulvestrant. In some embodiments, the endocrine therapy agent is a SERM. In some such embodiments, the SERM is selected from the group consisting of tamoxifen, raloxifene, toremifene, lasofoxifene, bazedoxifene and afimoxifene. In some such embodiments, the SERM is tamoxifen or raloxifene. In preferred embodiments, the endocrine therapy agent is letrozole or fulvestrant.

The endocrine therapy agent may be administered according to the standard of care per package insert or provided by the health care professionals. The term “package insert” refers to instructions customarily included in commercial packages of therapeutic products, that contain information about the indications, usage, dosage, administration, contraindications and/or warnings concerning the use of such therapeutic products.

In certain embodiments, the endocrine therapy agent is administered to the subject during the course of the treatment with PF-07104091. In certain embodiments, the first dose of the endocrine therapy agent is administered prior to administering the first dose of PF-07104091. In certain embodiments, the first dose of the endocrine therapy agent is administered on the same day as administering the first dose of PF-07104091. In certain embodiments, the first dose of the endocrine therapy agent is administered after the start of the treatment with PF-07104091.

In certain embodiments, prior to the administration of PF-07104091 to the subject, the subject has been previously treated with one or more lines of endocrine therapy.

In certain embodiments, prior to the administration of PF-07104091 to the subject, the subject is previously untreated, i.e. is treatment naïve.

In certain embodiments, prior to the administration of PF-07104091 to the subject, the subject has failed to achieve a sustained response after a prior therapy with a biotherapeutic or chemotherapeutic agent, i.e. is treatment experienced.

In certain embodiments, prior to the administration of PF-07104091 to the subject, the subject has been previously treated with chemotherapy, radiotherapy, and/or surgical resection.

In certain embodiments, prior to the administration of PF-07104091 to the subject, the subject has been previously treated with a CDK4/6 inhibitor.

In some embodiments of each of the methods, combinations and uses herein, the invention relates to neoadjuvant therapy, adjuvant therapy, first-line therapy, second-line therapy, or third-line or later therapy, in each case for treating cancer as further described herein. In each of the foregoing embodiments, the cancer may be localized, advanced or metastatic, and the intervention may occur at point along the disease continuum (i.e., at any stage of the cancer).

### EXAMPLES

The following examples are merely illustrative of the disclosure and should not be considered limiting the scope of the invention, as these examples and other equivalents thereof will become apparent to those skilled in the art in light of the present disclosure and the accompanying claims.
Example 1—Evaluation of PF-07104091 Modulation of Biomarker pRb Ser807/811 in CCNE1-Amplified Ovarian Cancer Cell Line and Anti-Proliferative Activity in CCNE1-Amplified Ovarian, HR+, HER2− Breast Cancer, and Rb Mutant SCLC Cell Lines

The cellular activity of PF-07104091 was evaluated by measuring phosphorylation of Rb at Ser807/811 following treatment of enriched G1/S phase CCNE1-amplified OVCAR3 ovarian carcinoma cells. In OVCAR3 cells, PF-07104091 demonstrated inhibition of pRb with a mean IC50 of 143.5 nM (Table 1).

Anti-proliferative activity was measured in cells treated for 7 days with PF-07104091 in OVCAR3 cells, Rb mutant NCI-H526 SCLC cells, and both HR-positive HER2-negative MCF7 and the subline MCF7-PalboR (resistant to palbociclib).

PF-07104091 inhibited MCF7 parental cells with an IC50 of 251.7 nM and MCF7-PalboR subline with an IC50 of 679.7 nM (Table 1). The MCF7-PalboR cell model is refractory to palbociclib-induced proliferative inhibition at doses up to 5 mM, which exceeds the concentration expected to inhibit CDK4 and CDK6. Proliferation of the OVCAR3 and NCI-H526 cell lines was inhibited by PF-07104091 with IC50 values of 118.5 nM and 136.4 nM, respectively (Table 1).

The effect of PF-07104091 on cell cycle distribution and DNA synthesis was determined following 72-hour treatment with 300 nM PF-07104091. Significant S phase reduction (p≤0.05 versus vehicle treated) was observed in OVCAR3, MCF7, and MCF7-PalboR while NCI-H526 trended towards significance (p=0.057). Significant G1 arrest was observed in OVCAR3 cells, while significant G2/M arrest was observed in HR-positive HER2-negative parental and palbociclib-resistant MCF7, and in NCI-H526 Rb mutant SCLC cells.

TABLE 1
Summary of Key In Vitro Pharmacologic
Properties of PF-07104091
Modulation of pRB Serine 807/811 by PF-07104091^{b}

| Cell Line | IC50 (nM) | SEM | n |
|---|---|---|---|
| OVCAR3 | 143.5 | 15.5 | 21 |

Proliferation Inhibition by PF-07104091^{c}

| Cell Line | IC₅₀ (nM) | SEM | n |
|---|---|---|---|
| OVCAR3 | 118.5 | 12.9 | 5 |
| MCF7 | 251.7 | 27.5 | 3 |
| MCF7-PalboR | 679.7 | 97.6 | 3 |
| NCI-H526 | 136.4 | 12.5 | 4 |

### Example 2—Inhibition of Proliferation of ER+ Human Breast Cancer (BC) Cells with PF-07104091 in Combination with Palbociclib

The combination effects of PF-07104091 with palbociclib were evaluated in a cell proliferation assay with MCF7 and T47D BC cell lines. Each compound exhibited potent single agent dose dependent growth inhibition in both cell models. Drug combinations were analyzed using the Dose Equivalence Principle, and the Loewe Volume score. When modeling synergy using the Dose Equivalence Principle, the Loewe Volume score for the combination compounds should be greater than the compound self-cross controls, and the Combination Index score for the combination compounds should be less than compound self-cross controls. The combinations of PF-07104091 with palbociclib demonstrated favorable profiles for these two metrics, Loewe Volume and CI, as compared to the self-cross controls in the MCF7 breast cancer cell line (Table 2) and the T47D breast cancer cell line (Table 3). Furthermore, the isobolograms set to an anti-proliferation threshold at 70% confirm a synergistic response. Based on the Loewe Volumes, CI values, and isobolograms, modest combination synergy is observed in these cell lines with the highest combinatorial benefit seen in the MCF7 model.

TABLE 2
Inhibition of Proliferation of ER+ Human Breast Cancer Cells
with PF-07104091 in Combination with Palbociclib in MCF7 Cells

| | | | | | | Loewe | |
|---|---|---|---|---|---|---|---|
| | | | Best CI | Best CI | Loewe | Volume | |
| Description | Combination Name | Best CI | Error | Level | Volume | Error | N |
| Combination | PF-07104091 × | 0.70 | 0.02 | 70 | 1.03 | 0.05 | 8 |
| | palbociclib | | | | | | |
| Self-cross | palbociclib × | 0.94 | 0.02 | 70 | −0.16 | 0.15 | 2 |
| | palbociclib | | | | | | |
| Control | PF-07104091 × | 1.04 | 0.03 | 70 | −0.08 | 0.08 | 4 |
| | PF-07104091 | | | | | | |

TABLE 3
Inhibition of Proliferation of ER+ Human Breast Cancer Cells
with PF-07104091 in Combination with Palbociclib in T-47D Cells

| | | | | | | Loewe | |
|---|---|---|---|---|---|---|---|
| | | | Best CI | Best CI | Loewe | Volume | |
| Description | Combination Name | Best CI | Error | Level | Volume | Error | N |
| Combination | PF-07104091 × | 0.72 | 0.02 | 70 | 0.41 | 0.06 | 8 |
| | palbociclib | | | | | | |
| Self-cross | palbociclib × | 0.96 | 0.13 | 70 | −0.25 | 0.18 | 2 |
| | palbociclib | | | | | | |
| Control | PF-07104091 × | 0.95 | 0.03 | 70 | 0.01 | 0.05 | 4 |
| | PF-07104091 | | | | | | |

### Example 3—Antitumor Activity of PF-07104091 in OVCAR3, MCF7, and NCI-H526 Tumor Models

### Methods

### In Vivo Efficacy Evaluation and Statistical Analysis

Female NSG (Jackson Lab) or Nu/nu (CRL) were subcutaneously implanted with tumor cells or fragments (8 mm³ to 27 mm³) into the dorsal region. For the MCF7 study, mice were supplemented with estrogen (sc implanted, 17β-estradiol, 0.36 mg, 90-day release pellet, Innovative Research of America, cat no. NE-121). Tumor volume and body weights were measured twice a week. Tumor volume was calculated using the [(Length×Width×Width)/2)] formula. TGI was calculated as 100*(1−ΔT/ΔC). The ΔC (ΔT) was obtained by subtracting the mean tumor burden in the vehicle (treated) group on the first day of treatment (Day 0) from the mean tumor burden in vehicle (treated) group on the assessment day. Statistical analysis was performed when the mean tumor volume in vehicle treated mice reached 950 to 1500 mm³ using t-test analysis with GraphPad Prism software 8.0.2 (GraphPad Software Inc, San Diego).

### In Vivo Efficacy Evaluation in the Cyclin E1 Amplified OVCAR3 Ovarian Cancer Tumor Model

To establish the OVCAR3 model, tumor cells (5×10⁶ cells/mouse with 50% Cultrex® Basement Membrane Matrix) were subcutaneously implanted in female NSG mice. The tumor bearing mice were randomly assigned to groups (n=10 per group) when the tumor volume reached between 136 mm³ and 209 mm³, and subsequently treated with: 1) vehicle (10% NMP/40% PEG300/50% 10 mM citrate buffer, pH 3.4); 2-4) PF-07104091 (batch No. 005) at 25, 75 and 175 mg/kg, BID, respectively. 175 mg/kg, po, BID is the maximum tolerated dose level in this model. PF-07104091 was administered (p.o.) BID (7 hr apart). All mice received treatment continuously for 21 days. TGI was assessed on Day 21 post first dose (or Day 66 post tumor cell implant).

### In Vivo Efficacy Evaluation in the HR+/HER2− MCF7 Breast Cancer Model

The MCF7 model was established by implanting donor tumor fragments into recipient mice. To establish the MCF7 donor mice, tumor cells (5×10⁶ cells/mouse with 50% Cultrex® Basement Membrane Matrix) were subcutaneously implanted in female NSG mice. Once reaching a range of 700 to 800 mm³, donor tumors were subsequently transplanted into secondary recipient mice for study expansion. When tumor volume reached a range between 100 mm³ to 291 mm³, the tumor bearing mice were randomly assigned to groups (n=10 per group) and dosed with: 1) vehicle (0.5% MC with 0.1% Tween 80 in water); 2-4) PF 07104091 (batch No. 006) at 25, 75 and 150 mg/kg BID, respectively. 150 mg/kg, po, BID is the maximum tolerated dose level in this model. PF 07104091 was administered (p.o.) BID (7 hr apart). TGI was assessed on Day 21 post first dose (or Day 42 post tumor fragment implant).

### In Vivo Efficacy Evaluation in the Rb Mutant NCI-H526 Small Cell Lung Cancer Model

To establish the NCI-H526 model, tumor cells (4×10⁶ cells/mouse with 50% Cultrex® Basement Membrane Matrix) were subcutaneously implanted in female Nu/nu mice. When the tumor volume reached a range between 63 mm³ and 138 mm³, the tumor bearing mice were randomly assigned to groups (n=15 for Group 1 and n=10 for Groups 2 to 8) and dosed with: 1) vehicle (0.5% MC with 0.1% Tween 80 in water); 2-4) PF 07104091 (batch No. 0014) at 25, 75 and 175 mg/kg BID, respectively; 5) Palbociclib (batch #GR08498) p.o. BID at 10 mg/kg; 6) Palbociclib (batch #GR08498) p.o. SID at 50 mg/kg. PF-07104091, Palbociclib at 10 mg/kg and CDK 2/4/6 inhibitor were administered (p.o.) BID (7 hr apart). TGI was assessed on Day 15 post first dose (or Day 20 post tumor cell implant).

### Results and Discussions

Antitumor efficacy following single agent PF-07104091 treatment was evaluated in the HR+, HER2− MCF7 BC, CCNE1-amplified OVCAR3 ovarian cancer, and Rb mutant NCI-H526 SCLC models.

In the OVCAR3 model, PF-07104091 PO BID treatment at 25, 75, and 175 mg/kg resulted in significant (p<0.1 versus vehicle) dose-dependent antitumor efficacy of 54%, 61% and 85% TGI (Tumor Growth Inhibition), respectively (Table 4). Similarly, in the MCF7 model, PF-07104091 PO BID treatment at 25, 75, and 150 mg/kg resulted in significant (p<0.01 versus vehicle) activity with TGI % values of 52%, 64% and 82%, respectively (Table 4). In the NCI-H526 model, PF-07104091 PO BID treatment at 175 mg/kg resulted in significant (p<0.01 versus vehicle) antitumor efficacy (78% TGI), but PF-07104091 was less efficacious at 25 and 75 mg/kg PO BID (44% and 45% TGI, respectively). By comparison, palbociclib administered either at 10 mg/kg BID or 50 mg/kg SID resulted in 33% and 22% TGI, respectively, in the NCI-526 model (Table 4).

TABLE 4
Antitumor Activity of PF-07104091 in Breast,
Ovarian and Small Cell Lung Cancer Models

| | | |
|---|---|---|
| | | Day 21 |
| Group | Treatment | TGI % |

OVCAR3 Model

| | | |
|---|---|---|
| 1 | Vehicle | — |
| 2 | PF-07104091 25 mg/kg BID | 54%** |
| 3 | PF-07104091 75 mg/kg BID | 61%** |
| 4 | PF-07104091 175 mg/kg BID | 85%** |

MCF7 Model

| | | |
|---|---|---|
| 1 | Vehicle BID | — |
| 2 | PF-07104091 25 mg/kg BID | 52%* |
| 3 | PF-07104091 75 mg/kg BID | 64%* |
| 4 | PF-07104091 150 mg/kg BID | 82%* |

NCI-H526 Model

| | | Day 15 |
|---|---|---|
| Group | Treatment | TGI % |
| 1 | Vehicle | — |
| 2 | PF-07104091 25 mg/kg BID | 44% |
| 3 | PF-07104091 75 mg/kg BID | 45% |
| 4 | PF-07104091 175 mg/kg BID | 78%* |
| 5 | Palbociclib 10 mg/kg BID | 33% |
| 6 | Palbociclib 50 mg/kg SID | 22% |

Vehicle = 10% NMP/40% PEG300/50% 10 mM citrate buffer, pH 3.4.
*= p < 0.01 versus vehicle; n = 10 to 15 mice/group;
**= p < 0.1 vehicle.
Source: PF-07104091_23Jan20_022132

### Example 4—Antitumor Efficacy of PF-07104091 in Combination with Palbociclib in T47D, HCC1428, and ST941 PBR Breast Cancer Tumor Models Methods

### In Vivo Efficacy Evaluation and Statistical Analysis

Female NSG mice (Jackson Lab) were subcutaneously implanted with fragment (27 mm³ to 64 mm³ size) into the dorsal region. All mice for T47D, HCC1428 and ST941 PBR studies were supplemented with 8.5 μg/mL estradiol water (β-Estradiol Sigma-Aldrich, cat #E2758-5G) and gave ad libitum till end of study. For the in vivo efficacy studies, tumor volume and body weights were measured twice a week. Tumor volume was calculated using the [(Length×Width×Width)/2)] formula. TGI was calculated as 100*(1−ΔT/ΔC). The ΔC (ΔT) was obtained by subtracting the mean tumor burden in the vehicle (treated) group on the first day of treatment (Day 0) from the mean tumor burden in vehicle (treated) group on the assessment day. Statistical analysis was performed using ANCOVA when the mean tumor volume in vehicle treated mice reached tumor cutoff size.

In Vivo Efficacy Evaluation of PF-07104091 in Combination with PF-07220060 or Palbociclib in the HR+/HER2− T47D Breast Cancer Model

The T47D model was established by implanting passage 3 tumor fragments into recipient mice. To establish the T47D donor mice, tumor cells (5×10⁶ cell/mouse with 50% Cultrex® Basement Membrane Matrix) were subcutaneously implanted in female NSG mice. Once reaching a range of 700 to 800 mm³, donor tumors were subsequently transplanted into secondary recipient mice for a study expansion. When tumor volume reached a range between 101 mm³ to 255 mm³, the tumor bearing mice were randomly assigned to groups (n=8 per group) and dosed with 1) vehicle (0.5% MC with 0.1% Tween 80 in water); 2) PF-07104091 at 150 mg/kg; 3) PF-07220060 at 60 mg/kg; 4) PD-0332991 at 10 mg/kg; 5) PF-07104091 at 150 mg/kg plus PF-07220060 at 60 mg/kg PF; 6) PF-07104091 at 150 mg/kg plus palbociclib at 10 mg/kg. PF-07104091 (lot 016), PF-07220060 (lot 019) and PD-0332991 (lot GR08498) were administered (PO.) BID (7 hr apart). All mice received treatment continuously until Day 41. TGI was assessed on Day 41 post first dose.

In Vivo Efficacy Evaluation of PF-07104091 in Combination with PF-07220060 or Palbociclib in the HR+/HER2− HCC1428 Breast Cancer Model

The HCC1428 model was established by implanting passage 4 tumor fragments into recipient mice. To establish the HCC1428 donor mice, tumor cells (5×10⁶ cell/mouse with 50% Cultrex® Basement Membrane Matrix) were subcutaneously implanted in female NSG mice. Once reaching a range of 700 to 800 mm³, donor tumors were subsequently transplanted into secondary recipient mice for a study expansion. When tumor volume reached a range between 100 mm³ to 272 mm³, the tumor bearing mice were randomly assigned to groups (n=10 per group) and dosed 1) vehicle (0.5% MC with 0.1% Tween 80 in water); 2) PF-07104091 at 150 mg/kg; 3) PF-07220060 at 60 mg/kg; 4) PD-0332991 at 10 mg/kg; 5) PF-07104091 at 150 mg/kg plus PF-07220060 at 60 mg/kg PF; 6) PF-07104091 at 150 mg/kg plus palbociclib at 10 mg/kg. PF-07104091 (lot 016), PF-07220060 (lot 019) and PD-0332991 (lot GR08498) were administered (PO.) BID (7 hr apart). All mice received treatment continuously until Day 42. TGI was assessed on Day 42 post first dose.

### Establishment of the In Vivo Palbociclib-Acquired Resistance HR+/HER2− BC PDX Model, ST941PBR

The ST941 PBR palbociclib resistant model was obtained from XENOSTART™ LLC, San Antonio, Texas. We established model in house by continuous treatment of the ST941 PBR tumor bearing mice with PD-0332991 at 50 mg/kg, PO QD plus fulvestrant at 10 mg/kg, SC twice in the first week, then weekly thereafter. To propagate donors for the TGI study, tumors were transplanted to recipient mice. One week after the re-implantation, the recipient mice received continuous treatment with palbociclib plus fulvestrant (dosing schedule described above). After 1 to 2 serial in vivo propagation with the same treatment regimen, the resistant tumors in the range of 700 to 800 mm³ were re-implanted for a study expansion. The remaining tumor fragments were viably frozen for future use.

To confirm resistance to treatment, ST941 PBR tumor bearing mice (p12) received palbociclib (50 mg/kg) plus fulvestrant (10 mg/kg) using the same treatment schedule as described above.

In Vivo Efficacy Evaluation of PF-07104091 in Combination with PF-07220060 or Palbociclib in the ST941PBR PDX Model

The live donor mice were established by implanting ST941 PBR (p11) tumor fragments. Once reaching a range of 700 to 800 mm³, the donor tumors were subsequently transplanted into secondary recipient mice for a study expansion. To maintain the resistant clones, the ST941 PBR tumor bearing mice were treated with PD-0332991 at 50 mg/kg, PO. QD plus fulvestrant at 10 mg/kg, SC twice in the first week, then weekly afterwards for 10 weeks. The treatment was initiated one week after the implant until day of randomization for study enrollment. When tumor volume reached a range between 121 mm³ to 228 mm³, the tumor bearing mice were randomly assigned to groups (n=10 per groups) and dosed with 1) vehicle (0.5% MC with 0.1% Tween 80 in water); 2) PF-07104091 at 150 mg/kg; 3) PF-07220060 at 60 mg/kg; 4) PD-0332991 at 10 mg/kg; 5) PF-07104091 at 150 mg/kg plus PF-07220060 at 60 mg/kg PF; 6) PF-07104091 at 150 mg/kg plus palbociclib at 10 mg/kg; 7) PD-0332991 at 10 mg/kg plus fulvestrant at 10 mg/kg; 8) PD-0332991 at 50 mg/kg plus fulvestrant at 10 mg/kg. PF-07104091 (lot 016), PF-07220060 (lot 019), PF-06873600 (lot 022) and PD-0332991 (lot GR08498) at 10 mg/kg were administered (PO) BID (7 hr apart); PD-0332991 (lot GR08498) at 50 mg/kg were administered (PO) QD and Fulvestrant at 10 mg/kg, SC twice in the first week, then weekly afterwards. All mice received treatment continuously until Day 28. TGI was assessed on Day 28 post first dose.

### Results and Discussions

PF-07104091 was also evaluated as a single agent and in combination with palbociclib in three HR+, HER2− BC models, T47D, HCC1428, and palbociclib-resistant ST941 PBR PDX. Treatments were administered PO, BID at mg/kg/dose indicated in Table 5 (except the 50 mg/kg QD palbociclib group and SC fulvestrant twice the first week and weekly thereafter). There were no significant body weight changes or other clinical observations noted throughout the treatment period in any of the 3 models (clinical pathology not conducted).

In the T47D model, PF-07104091 and palbociclib single agent treatment showed significant TGI efficacy versus vehicle (p<0.05). PF-07104091 plus palbociclib (TGI 100%) displayed significantly enhanced efficacy versus either monotherapy (p<0.05 vs either single agent).

In the HCC1428 model, PF-07104091 and palbociclib single agent treatment also showed significant TGI efficacy versus vehicle (p<0.05). PF-07104091 plus palbociclib treatment displayed significantly enhanced efficacy (TGI 106%), versus either palbociclib (TGI 44%) or PF-07104091 (TGI 95%) monotherapies (Table 5).

In the palbociclib resistant ST941 PBR PDX model, palbociclib (10 mg/kg BID or 50 mg/kg QD) treatment in combination with fulvestrant showed significant but minimum response (30% and 26% TGI, respectively, versus vehicle), confirming acquired resistance (Table 5). PF-07104091 in combination with palbociclib resulted in significant TGI (49%) versus vehicle or either PF-07104091 (TGI 34%) or palbociclib monotherapies.

TABLE 5
PF-07104091 Activity as a Single Agent or In Combination
with CDK4 Inhibitor PF-07220060 or Palbociclib in
HR+, HER2− Breast Cancer Models (T47D and
HCC1428) and Palbociclib-Resistant ST941PBR PDX

| | | T47D | HCC1428 | ST941PBR |
|---|---|---|---|---|
| | | Day 41 | Day 42 | Day 28 |
| Group | Treatment | TGI % | TGI % | TGI % |
| 1 | Vehicle | — | — | — |
| 2 | PF-07104091 | 82* | 95* | 34* |
| | 150 mg/kg BID | | | |
| 4 | Palbociclib | 72* | 44* | 15 |
| | 10 mg/kg BID | | | |
| 6 | PF-07104091 | 100*tα | 106*tα | 49*tα |
| | 150 mg/kg | | | |
| | BID + Palbociclib | | | |
| | 10 mg/kg BID | | | |
| 7 | Palbociclib | NA | NA | 30* |
| | 10 mg/kg | | | |
| | BID + Fulvestrant | | | |
| | 10 mg/kg (2x week | | | |
| | 1, then Q7Dx4) | | | |
| 8 | Palbociclib | NA | NA | 26* |
| | 50 mg/kg QD + | | | |
| | Fulvestrant | | | |
| | 10 mg/kg (2x week | | | |
| | 1, then Q7Dx4) | | | |

TGI was assessed post first dose on Day 41 for T47D (n = 7 to 8/group), Day 42 for HCC1428 (n = 10/group), and Day 28 for ST941PBR (n = 9 to 10/group). Vehicle = 0.5% MC with 0.1% Tween 80 in water. Statistical analysis was performed using ANCOVA.
*indicates p < 0.05 vs. vehicle;
t: indicates p < 0.05 vs. PF-07104091 treatment;
α: indicates p < 0.05 vs palbociclib.

### Example 5—A Phase 1/2a Study of PF-07104091 as a Single Agent and in Combination with Palbociclib and/or Endocrine Therapy (ET)

### Study Design

This is an ongoing Phase 1/2a, open label, multi center, multiple dose, dose escalation, safety, PK and PD study of PF-07104091 administered as a single agent in sequential dose levels, and then in combination with a CDK4/6 inhibitor (palbociclib), and in combination with palbociclib and letrozole is conducted in selected tumor indications. Successive cohorts of participants will receive escalating doses of PF-07104091 starting from 75 mg orally BID.

This clinical study will be comprised of 2 parts (Parts 1 and 2). Part 1A will be the dose escalation part with single agent PF-07104091. Intraparticipant dose escalation will not be permitted. Part 1B will be dose finding with PF-07104091 in combination with palbociclib and fulvestrant and Part 1C will be a dose finding with PF-07104091 in combination with palbociclib and letrozole). Intraparticipant dose escalation will not be permitted. Part 2 will be dose expansion and include study of PF-07104091 as a single agent in Arms A, B and C and PF-071041 in combination with palbociclib and endocrine therapy in Arms D and E (fulvestrant) or Arm F (letrozole).

The effect of food on the PK of PF-07104091 will be assessed in participants who may be enrolled in Part 1 and/or Part 2.Part 1A will determine the maximum tolerated dose (MTD) and recommended Phase 2 dose (RP2D) in sequential dose escalation safety cohorts for PF-07104091 as a single agent in participants with advanced or metastatic SCLC, advanced platinum resistant ovarian cancer, locally recurrent/advanced or metastatic TNBC, women of any menopausal status and men with HR+/HER2− advanced or metastatic breast cancer, and in advanced or metastatic NSCLC. Any given PF-07104091 dose in Part 1A may be escalated or de-escalated to the next higher or lower dose, or to an intermediate dose level, depending on emerging safety findings.

Parts 1B and 1C will include participants with women of any menopausal status and men with HR-positive HER2-negative advanced or metastatic breast cancer mBC and will estimate the MTD/RP2D of PF-07104091 in combination with palbociclib and fulvestrant or letrozole, respectively.

Part 2 will evaluate the doses selected (MTD/RP2Ds) for PF-07104091 as single agent and in combination therapy depending on clinical observations from Part 1, in dose expansion arms as follows:

(a) Part 2 Arm A will include participants with SCLC who have received at least 1 systemic anti-cancer therapy containing platinum.

(b) Part 2 Arm B will include participants with ovarian cancer. Participants will be enrolled based on tumor CCNE1 gene amplification status, as previously determined locally by using a next-generation sequencing (NGS)-, PCR- or FISH-based tumor tissue test (unless not known and/or not performed/not available) that is either approved by the FDA or analytically validated and performed in a CLIA-certified laboratory.

(c) Part 2 Arm C will include participants with TNBC.

(d) Part 2 Arm D will include participants with HR-positive HER2-negative advanced or mBC (second plus line setting post CDK 4/6).

(e) Part 2 Arm E will include participants with HR-positive HER2-negative advanced or mBC (second plus line setting, CDK 4/6 naive).

(f) Part 2 Arm F will include participants with HR-positive HER2-negative advanced or mBC (first line setting, CDK 4/6 naive).

At the conclusion of Part 1A, PF-07104091 evaluation in dose expansion Part 2 Arms A, B and C may be initiated based on preceding safety and clinical observations. At the conclusion of Part 1B, PF-07104091 evaluation in dose expansion Part 2 Arms D and E may be initiated based on preceding safety and clinical observations. At the conclusion of Part 1C, PF-07104091 evaluation in dose expansion Part 2 Arm F may be initiated based on preceding safety and clinical observations.

PF-07104091 will initially be administered orally BID on a continuous basis under empty stomach conditions. Oral PF-07104091 was administered with at least 8 oz (240 mL) of water on an empty stomach. No food or liquids other than water was consumed for 2 hours before and 1 hour following each dose throughout the study.

If emerging PK data from initial dose levels in Part 1A demonstrate the feasibility of QD dosing, the dose escalation may continue with QD dosing. If preliminary PK data from initial dose levels suggest the need for more frequent daily dose level, the dose escalation may continue with increases in the total daily dose up to a maximum of TID. A cycle is defined as 28 days, regardless of missed doses or dose delays.

The evaluation of an alternative dosing regimen (e.g., QD or TID) may be considered during the course of dose escalation or after determination of the MTD/RP2D for the BID regimen based on emerging and available preliminary clinical data, including safety/tolerability, laboratory, PK and PD findings. A Bayesian Logistic Regression Model (BLRM) along with the escalation with overdose control (EWOC) criteria will be used in both Parts 1A and 1B to guide the dose escalation and to determine the MTD/RP2D of PF-07104091 as a single agent as well as PF-07104091 in combination with palbociclib and endocrine therapy (fulvestrant or letrozole). To identify the optimal dosing regimen (e.g., different starting dose or different schedule) of PF-07104091 in combination with letrozole or fulvestrant to be identified, an additional dosing regimen(s) determined to be safe in Parts 1B and 1C may be investigated in Part 2 Arms D, E, and F (HR-positive HER2-negative breast cancer cohorts). Alternative combination dosing strategies may be explored in accordance with BLRM/EWOC.

PF-07104091 will be provided as tablets for oral administration. The 25 mg and 125 mg tablets will be supplied in separate bottles and labeled according to local regulatory requirements. Palbociclib will be provided as capsules for oral administration. The 75 mg, 100 mg, and 125 mg capsules will be supplied in separate bottles and labeled according to local regulatory requirements. Letrozole will be provided as 2.5 mg tablets for oral administration in either blister packs or bottles and labeled according to local regulatory requirements. Fulvestrant will be provided as 250 mg/5 mL prefilled syringes in a kit containing 2 syringes (500 mg dose) for intramuscular injection.

### Starting Dose for Single Agent Dose Escalation (Part 1A)

The selection of the starting dose and regimen was based on the nonclinical toxicology and PK results in accordance with the ICH S9 Guidance. Results from nonclinical toxicity studies indicate the STD10 of PF-07104091 in mouse was 150 mg/kg after BID oral administration. The human equivalent dose for the STD10 in mouse is approximately 729 mg BID (assuming a body weight of 60 kg). Per ICH-S9 guidance, one-tenth of the STD10 can be considered the appropriate starting dose. Given the predicted human t % of approximately 4 hours, the starting dose has been selected to be 75 mg BID. The human AUC0-12 hr of PF-07104091 at the proposed starting oral dose of 75 mg BID is predicted to be ˜6.5% of the observed AUC0-12 hr at the STD10 in mouse (150 mg/kg BID) and ˜80% of the observed AUC0-12 hr at the HNSTD in dog (15 mg/kg BID).

### Starting Dose for Combination Dose Finding (Part 1B and 1C)

The dose finding evaluation of PF-07104091 in combination with palbociclib and endocrine therapy (fulvestrant or letrozole) is planned to be initiated at a dose of PF-07104091 not higher than the highest dose of PF-07104091 as a single agent previously determined to be safe (Part 1A), as guided by the BLRM and EWOC criteria, based on potential overlapping toxicity considerations and DDI assessment, and supported by emerging clinical data including available safety/tolerability, PK, and PD findings. Study participants will be evaluated for the safety and tolerability of the combination; the PF-07104091 dose used in the next cohort may remain the same, escalated, or de-escalated as guided by emerging clinical data and BLRM. However, PF-07104091 dose in combination cannot be higher than the MTD/RP2D for PF-07104091 as a single agent in Part 1A. The starting dose of palbociclib will be at 125 mg orally daily although a lower starting dose of palbociclib (e.g., 100 or 75 mg) may be explored based on emerging data, potential overlapping toxicity considerations and drug-drug interaction (DDI) assessment. Dose of letrozole will be 2.5 mg orally daily. Dose of fulvestrant will be 500 mg on Days 1, 15, and 29 (Cycle 2 Day 1), and every 4 weeks thereafter.

### Intervention Groups and Duration

Part 1A, PF-07104091 will be evaluated as a single agent. Each cycle will be 28 days in duration. PF-07104091 will be administered BID orally on days 1 to 28 of each cycle. The proposed doses, schedule, and PK time points may be revised and amended during the study based on the emerging safety and PK data.

Part 1B, PF-07104091 will be evaluated in combination therapy with palbociclib and fulvestrant. Each cycle will be 28 days in duration. PF-07104091 will be administered BID orally on days 1 to 28 of each cycle. Palbociclib will be administered orally once daily on intermittent dosing on a 21-day schedule (Day 1 to Day 21) followed by a 7-day dosing holiday (Day 22 to Day 28; i.e., 3 weeks on/1 week off). Fulvestrant will be administered by intramuscular injection on Days 1, 15, and 29 (Cycle 2 Day 1), and then every 4 weeks thereafter.

Part 1C: PF-07104091 will be evaluated in combination therapy with palbociclib and letrozole. Each cycle will be 28 days in duration. PF-07104091 will be administered BID orally on days 1 to 28 of each cycle. Palbociclib will be administered orally once daily on intermittent dosing on a 21-day schedule (Day 1 to Day 21) followed by a 7-day dosing holiday (Day 22 to Day 28; i.e., 3 weeks on/1 week off). Letrozole will be administered once a day orally according to US package insert.

### Criteria for Dose Escalation

The objective of Part 1 of the study is to establish the MTD/RP2D. The MTD for PF-07104091 as a single agent and for combination corresponds to the highest dose with a true DLT rate in the target interval (0.16, 0.33). This definition of MTD will be used to guide the dose escalation of PF-07104091 as a single agent and in combination with palbociclib and endocrine therapy (fulvestrant or letrozole) in Part 1 of the study, following the Bayesian methodology with EWOC principle. The use of the EWOC principle limits the risk that a potential next dose will exceed the MTD. The final estimate of the MTD(s) and/or RP2D(s) should also satisfy the EWOC condition.

Initial dose levels in Part 1A are provided in Table 6 and intermediate dose levels may be considered as indicated.

TABLE 6
PF-07104091 Dose Levels (Immediate Release Formulation)

| | | |
|---|---|---|
| Dose | | Total |
| Level (DL) | Dose | Daily Dose |

| | | | | |
|---|---|---|---|---|
| DL1 | 50 | mg BID | 100 | mg |
| DL1** | 75 | mg BID | 150 | mg |
| DL2 | 150 | mg BID | 300 | mg |
| DL3 | 300 | mg BID | 600 | mg |
| DL4 | 500 | mg BID | 1000 | mg |

| | |
|---|---|
| DL5 and | Escalation/de-escalation to |
| beyond | continue too the MTD/RP2D |

**Starting dose DL1.

The proposed doses, schedule(s), may be reconsidered or amended during the study based on the emerging safety and PK data. Additional doses explored to date are 375 and 225 mg BID.

The dose escalation will stop when the MTD/RP2D is determined or stopping criteria are met.

DLTs will be assessed through Cycle 1 (28-day cycle with Day 29 laboratory assessments). For the purpose of dose escalation, the DLT observation period will be during the first cycle (28 days after the start of study treatment) in each participant. Severity of AEs will be graded according to Common Terminology Criteria for Adverse Events (CTCAE) version 5.0. For the purpose of dose escalation and dose finding, any of the following AEs with the associated parameters that occur in the first cycle of treatment or within 28 days after the start of the study treatment which are attributable to PF-07104091 single agent or in combination with the other agents will include hematologic or non-hematologic DLTs.

### Part 2 PF-07104091 Single Agent and Combination Dose Expansion

Part 2 dose expansion is an open-label, multi-center, non-randomized study to assess the preliminary anti-tumor activity and the safety and tolerability of PF-07104091. PF-07104091 will be administered at a dose not exceeding the MTD/RP2D in 28-day cycles as a single agent in 3 separate dose single agent expansion arms and in 3 separate combination dose expansion arms. The PF-07104091 dose as a single agent and combinations could be further optimized (remain the same, escalated or de-escalated) in Part 2 depending on emerging data if indicated. Approximately 30 participants (approximately 40 participants in Part 2 Arm B) will be enrolled in each of the study arms.

### Intervention Groups and Duration

Part 2 Arm A, B and C: PF-07104091 will be evaluated as a single agent at a dose not exceeding the MTD/RP2D. Each cycle will be 28 days in duration. PF-07104091 will be administered BID orally on days 1 to 28 of each cycle. The proposed doses, schedule, and PK time points may be revised and amended during the study based on the emerging safety and PK data.

Part 2 Arm D and E: PF-07104091 will be evaluated in combination therapy with palbociclib and fulvestrant at a dose not exceeding the MTD/RP2D. Each cycle will be 28 days in duration. PF-07104091 will be administered BID orally on days 1 to 28 of each cycle. Palbociclib will be administered orally once daily intermittent dosing on a 21-day schedule (Day 1 to Day 21) followed by a 7-day dosing holiday (Day 22 to Day 28; i.e., 3 weeks on/1 week off). Fulvestrant will be administered by intramuscular injection on Days 1, 15 and 29 (Cycle 2 Day 1), and then 4 weeks thereafter, according to US package insert.

Part 2 Arm F: PF-07104091 will be evaluated in combination therapy with palbociclib and letrozole at a dose not exceeding the MTD/RP2D. Each cycle will be 28 days in duration. PF-07104091 will be administered BID orally on Days 1 to 28 of each cycle. Palbociclib will be administered orally once daily intermittent dosing on a 21-day schedule (Day 1 to Day 21) followed by a 7-day dosing holiday (Day 22 to Day 28; i.e., 3 weeks on/1 week off). Letrozole will be administered once a day orally according to US package insert.

The proposed doses, schedule, and PK time points may be revised and amended during the study based on the emerging safety and PK data.

### Food Effects on PF-07104091 PK Substudy

The effect of food on the PK of PF-07104091 will be assessed in at least 6 participants who may be enrolled in Part 1 and/or Part 2 at a dose not exceeding the single agent MTD/RP2D. Each participant will serve as his or her own control. A one-way crossover design will be utilized to assess the steady state PK of PF-07104091 under the fasted and fed conditions on Cycle 1 Day 15 and Cycle 1 Day 16, respectively.

### Study Population

### Inclusion Criteria

The following eligibility criteria are designed to select participants for whom participation in the study is considered appropriate. Participants are eligible to be included in the study only if all of the following criteria apply.
1. Females and/or male participants age 18 years.
2. (a) Part 1:

Breast cancer: Participants with HR-positive HER2-negative advanced or metastatic breast cancer (third line plus setting) (histologically or cytologically proven).

TNBC: Participants with locally recurrent/advanced or metastatic TNBC who have received up to 3 prior lines of chemotherapy in the advanced or metastatic setting.

Ovarian cancer: Participants with advanced platinum resistant epithelial ovarian cancer (EOC)/fallopian tube cancer/primary peritoneal cancer (PPC) (histologically or cytologically proven) who have received at least 1 systemic anti-cancer therapy containing a platinum analog.

Part 1A only:

SCLC: Participants with cytological diagnosis of advanced/metastatic SCLC.

NSCLC: Participants with or cytological diagnosis of advanced/metastatic NSCLC.

Part 1B and 1C only:

Breast cancer: Participants with HR-positive HER2-negative advanced or metastatic breast cancer (second line plus setting) (histologically or cytologically proven).

(b) Part 2:

Arm A: SCLC—Participants with cytological diagnosis of advanced/metastatic SCLC. Participants must have received at least 1 systemic anti-cancer therapy containing platinum.

Arm B: Ovarian cancer: Participants with advanced platinum resistant epithelial ovarian cancer (EOC)/fallopian tube cancer/primary peritoneal cancer (PPC) (histologically or cytologically proven) who have received at least 1 systemic anti-cancer therapy containing a platinum analog.

Arm C: TNBC: Participants with locally recurrent/advanced or metastatic TNBC who have received up to 2 prior lines of chemotherapy in the advanced or metastatic setting.

Arm D: HR-positive HER2-negative advanced or metastatic breast cancer—Participants with HR-positive HER2-negative advanced or metastatic breast cancer after prior ET-CDK4/6 inhibitor therapy (histologically or cytologically proven).

Arm E: HR-positive HER2-negative advanced or metastatic breast cancer—Participants with HR-positive HER2-negative advanced or metastatic breast cancer after prior ET (histologically or cytologically proven). No prior fulvestrant, CDK4/6, mTOR or PI3K inhibitor.

Arm F: HR-positive HER2-negative advanced or metastatic breast cancer—Participants with HR-positive HER2-negative advanced or metastatic breast cancer (first-line setting) (histologically or cytologically proven).

3. Participants entering the study in the expansion cohort have at least one measurable lesion as defined by RECIST version 1.1 that has not been previously irradiated).

4. ECOG PS 0 or 1.

5. Adequate Bone Marrow Function, including: ANC≥1,500/mm³ or ≥1.5×109/L; Platelets≥100,000/mm³ or ≥100×109/L; Hemoglobin≥9 g/dL.

6. Adequate Renal Function, including: Estimated creatinine clearance 50 mL/min could be acceptable as calculated using the method standard for the institution.

In equivocal cases, a 24-hour urine collection test can be used to estimate the creatinine clearance more accurately.

7. Adequate Liver Function, including: Total serum bilirubin≤1.5×ULN unless the participant has documented Gilbert syndrome; AST and ALT≤2.5×ULN; ≤5.0×ULN if there is liver involvement by the tumor; Alkaline phosphatase≤2.5×ULN (≤5×ULN in case of bone metastasis).

8. Resolved acute effects of any prior therapy to baseline severity or CTCAE Grade≤1 except for AEs not constituting a safety risk by investigator judgment.

9. Participants who are willing and able to comply with all scheduled visits, treatment plan, laboratory tests, lifestyle considerations, and other study procedures.

10. Capable of giving signed informed consent, which includes compliance with the requirements and restrictions listed in the ICD and in this protocol.

### Exclusion Criteria

1. Participants with known symptomatic brain metastases requiring steroids. Participants with previously diagnosed brain metastases are eligible if they have completed their treatment and have recovered from the acute effects of radiation therapy or surgery prior to study entry, have discontinued corticosteroid treatment for these metastases for at least 4 weeks and are neurologically stable for 3 months (requires MRI confirmation). Participants with previously diagnosed CNS disease are eligible if they have completed their treatment and have recovered from the acute effects of therapy prior to study entry, and are neurologically stable for 3 months (requires MRI confirmation).

2. Participants with any other active malignancy within 3 years prior to enrollment, except for adequately treated basal cell or squamous cell skin cancer, or carcinoma in situ.

3. Major surgery within 3 weeks prior to study entry.

4. Radiation therapy within 3 weeks prior to study entry.

5. Systemic anti-cancer therapy within 4 weeks prior to study entry (6 weeks for mitomycin C or nitrosoureas) or 5 half-lives (whichever is shorter) of the agent(s) prior to receive the study intervention treatment is required.

6. Prior irradiation to >25% of the bone marrow (see Section 10.9 Bone Marrow Reserve in Adults).

7. Participants with active, uncontrolled bacterial, fungal, or viral infection, including HBV, HCV, and known HIV or AIDS related illness. In equivocal cases, participants whose viral load is negative, may be eligible. HIV seropositive participants who are healthy and low risk for AIDS-related outcomes could be considered eligible. Eligibility criteria for HIV positive participants should be evaluated and discussed with sponsor's medical monitor, and will be based on current and past CD4 and T cell counts, history (if any) of AIDS defining conditions (eg, opportunistic infections), and status of HIV treatment. Also the potential for drug-drug interactions will be taken into consideration.

8. COVID-19/SARS-CoV2: This protocol excludes participants with active infections, as noted above. While SARS-CoV2 testing is not mandated for entry into this protocol, testing should follow local clinical practice standards. If a participant has a positive test result for SARS-CoV2 indicating current infection, is known to have asymptomatic, current infection or is suspected of having current SARS-CoV2 infection, he/she is excluded. A positive test result may be repeated after 72 hours and the patient may be eligible if repeat testing is negative and the patient is asymptomatic. A participant without active SARS-CoV2 infection with a positive test (eg, antibody test) indicating past, resolved infection or vaccination is still eligible.

9. Baseline 12-lead ECG that demonstrates clinically relevant abnormalities that may affect participant safety or interpretation of study results (eg, baseline QTc interval>470 msec, complete LBBB, signs of an acute or indeterminate-age myocardial infarction, ST-T interval changes suggestive of active myocardial ischemia, second- or third-degree AV block, or serious bradyarrhythmias or tachyarrhythmias). If the baseline uncorrected QT interval is >470 msec, this interval should be rate-corrected using the Fridericia method and the resulting QTcF should be used for decision making and reporting. If QTc exceeds 470 msec, or QRS exceeds 120 msec, the ECG should be repeated 2 more times and the average of the 3 QTc or QRS values should be used to determine the participant's eligibility. Computer-interpreted ECGs should be overread by a physician experienced in reading ECGs before excluding participants. Cases must be discussed in detail with sponsor's medical monitor to judge eligibility.

10. Any of the following in the previous 6 months: myocardial infarction, long QT syndrome, Torsade de Pointes, arrhythmias (including sustained ventricular tachyarrhythmia and ventricular fibrillation), serious conduction system abnormalities (eg, bifascicular block such as RBBB with left anterior or posterior hemiblock, third-degree heart block), unstable angina, coronary/peripheral artery bypass graft, symptomatic CHF, New York Heart Association class III or IV, cerebrovascular accident, transient ischemic attack, symptomatic pulmonary embolism, and/or other clinical significant episode of thromboembolic disease. Ongoing cardiac dysrhythmias of NCI CTCAE Grade 2, atrial fibrillation of any grade (Grade 2 in the case of asymptomatic lone atrial fibrillation). If a participant has a cardiac rhythm device/pacemaker placed and QTcF>470 msec, the participant can be considered eligible. Participants with cardiac rhythm device/pacemaker must be discussed in detail with sponsor's medical monitor to judge eligibility.

11. Anticoagulation with vitamin K antagonists or factor Xa inhibitors is not allowed. Anticoagulation with subcutaneous heparin is allowed. Participants on anticoagulants for underlying medical condition may be considered after discussion with the sponsor.

12. Hypertension that cannot be controlled by medications (eg, >150/90 mmHg) despite optimal medical therapy.

13. Participation in other studies involving investigational drug(s) within 2 weeks prior to study entry.

14. Known or suspected hypersensitivity to active ingredient/excipients in PF-07104091.

15. Active inflammatory gastrointestinal disease, chronic diarrhea, known diverticular disease or previous gastric resection or lap band surgery. Gastroesophageal reflux disease under treatment with proton pump inhibitors is allowed (assuming no drug interaction potential).

16. Participants with advanced/metastatic, symptomatic, visceral spread, that are at risk of life-threatening complications in the short term (including participants with massive uncontrolled effusions [pleural, pericardial, peritoneal], pulmonary lymphangitis, and over 50% liver involvement).

17. Participants with an indwelling catheter that has an external component such as those used for drainage of effusion(s) or central venous catheter that is externally exposed (eg, peripherally inserted central catheter (PICC) line). Indwelling catheters that are fully internalized (eg, PORTACATH®) are permitted.

18. Previous high dose chemotherapy requiring stem cell rescue.

19. Known abnormalities in coagulation such as bleeding diathesis, or treatment with anticoagulants precluding intramuscular injections of goserelin (if applicable).

20. 20. Known or suspected hypersensitivity to active ingredient/excipients of PF-07104091, palbociclib (or equivalent agent to induce chemical menopause).

21. Current use or anticipated need for food or drugs that are known strong CYP3A4/5 or UGT1A9 inhibitors, including their administration within 5 half-lives of the CYP3A4/5 or UGT1A9 inhibitor prior to first dose of investigational product.

22. Current use or anticipated need for drugs that are known strong CYP3A4/5 or UGT1A9 inducers, including their administration within 5 half-lives of the CYP3A4/5 or UGT1A9 inducer prior to the first dose of investigational product.

23. Current use or anticipated need for drugs that are known sensitive UGT1A1 substrates with narrow therapeutic index (eg SN-38 [active metabolite of irinotecan], irinotecan, belinostat). Any questions regarding the exclusion of concomitant medications should be directed to the sponsor.

24. Serum pregnancy test (for females of childbearing potential) positive at screening.

25. Other medical or psychiatric condition including recent (within the past year) or active suicidal ideation/behavior or laboratory abnormality that may increase the risk of study participation or, in the investigator's judgment, make the participant inappropriate for the study.

26. Investigator site staff or Pfizer employees directly involved in the conduct of the study, site staff otherwise supervised by the investigator, and their respective family members.

27. Prior treatment with the following for Part 2 Arm E and Arm F are excluded:
For Part 2 Arm E: Prior treatment with any CDK inhibitor, or fulvestrant, or everolimus, or any agent whose mechanism of action is to inhibit the PI3K-mTOR pathway.
For Part 2 Arm F:
Prior neoadjuvant or adjuvant treatment with a non-steroidal aromatase inhibitor (ie, anastrozole or letrozole) with disease recurrence while on or within 12 months of completing treatment.
Prior treatment with any CDK4/6 inhibitor.

### Dose Modification

Every effort should be made to administer study intervention on the planned dose and schedule. In the event of significant toxicity, dosing may be delayed and/or reduced as described below. In the event of multiple toxicities, dose modification should be based on the worst toxicity observed (and attribution for the combination treatment—use if a combination is to be tested). Participants are to be instructed to notify investigators at the first occurrence of any adverse symptom.

Dose modifications may occur in one of three ways: (1) Within a cycle: dosing interruption until adequate recovery and dose reduction, if required, during a given treatment cycle; (2) Between cycles: next cycle administration may be delayed due to persisting toxicity when a new cycle is due to start; or (3) In the next cycle: dose reduction may be required in a subsequent cycle based on toxicity experienced in the previous cycle.

### Dosing Interruptions

With respect to study intervention, participants experiencing the following adverse events should have their treatment interrupted: (1) DLT in Cycle 1 (DLT observation period of 28 days; or (2) AEs meeting DLT criteria after the DLT observation period. Appropriate follow-up assessments should be done until adequate recovery occurs as assessed by the investigator.

Doses may be held up to 4 weeks until toxicity resolution. Depending on when the adverse event resolved, a treatment interruption may lead to the participant missing all subsequent planned doses within that same cycle or even to delay the initiation of the subsequent cycle.

Following dosing interruption or cycle delay due to toxicity, the PF-07104091 dose may need to be reduced when treatment is resumed.

### Dose Reduction

Following dosing interruption or cycle delay due to toxicity, the PF-07104091 dose may need to be reduced when treatment is resumed. No specific dose adjustments are recommended for Grade 1/2 treatment-related toxicity. However, investigators should always manage their participants according to their medical judgment based on the particular clinical circumstances. Dose reduction of PF-07104091 by 1 and, if needed, 2 dose levels (2), or intermediate dose levels, will be allowed depending on the type and severity of toxicity encountered. Participants requiring more than 2 dose reductions will be discontinued from the treatment and entered into the follow-up phase, unless otherwise agreed between the investigator and the sponsor. All dose modifications/adjustments must be clearly documented in the participant's source notes and CRF.

Once a dose has been reduced for a given participant, all subsequent cycles should be administered at that dose level, unless further dose reduction is required. Intraparticipant dose re-escalation is not allowed. Participants experiencing a DLT may resume dosing at the next lower dose level (if applicable) once adequate recovery is achieved, and in the opinion of the investigator and sponsor, the participant is benefiting from therapy.

### Pharmacokinetic Analysis of PF-07104091

Blood samples will be collected for measurement of plasma concentrations of PF-07104091 as specified in the protocol. Samples will be used to evaluate the PK of PF-07104091. Samples collected for analyses of PF-07104091 plasma concentration may also be used to evaluate safety or efficacy aspects related to concerns arising during or after the study, for metabolite identification and/or evaluation of the bioanalytical method, or for other internal exploratory purposes.

For all participants participating in the food effect on PF-07104091 PK substudy, a single urine sample (“urine blank”) will be collected on Cycle 1 Day 1 prior to morning PF-07104091 administration, and multiple samples will be collected for 12 hours after morning PF-07104091 dosing on Cycle 1 Day 15 to measure PF-07104091 concentrations.

### Pharmacodynamics

Pharmacodynamic parameters will be evaluated in this study. Pharmacodynamic effects of PF-07104091 will be assessed in de novo (fresh) tumor biopsies, skin punches, and peripheral blood samples in this study to understand target engagement and modulation by the study intervention. As part of understanding the pharmacodynamics of the study intervention, samples may be used for evaluation of the bioanalytical method, as well as for other exploratory purposes.

### Statistical Methods

### Primary Endpoint(s)

For Part 1A, Part 1B, and Part 1C, determination of MTD/RP2D will be performed using the Per-protocol analysis set (participants evaluable for MTD).

### Bayesian Adaptive Approach

The Part 1A dose escalation, Part 1B and Part 1C dose finding part of the study will be guided by a Bayesian analysis of DLT data for PF-07104091 as a single agent and in combination with palbociclib and endocrine therapy (fulvestrant or letrozole). Toxicity is modelled using a 2-parameter BLRM for the probability of a participant to experience a DLT at the given dose of PF-07104091 as single agent (Part 1A) and a 10-parameter BLRM will be used to model the dose toxicity relationship of PF-07104091 given in combination with palbociclib and endocrine therapy (fulvestrant or letrozole) (Part 1B and 1C). Dosing decisions are guided by the EWOC principle. A dose may only be used for newly enrolled participants if the risk of excessive toxicity at that dose is less than 25%.

### Part 1 Dose Escalation

Approximately 40-50 participants are expected to be enrolled in the Part 1A single agent dose escalation portion and the Part 1B and 1C dose finding portions of the study. The total number of participants will depend on the number of dose levels needed to determine the MTD and the actual number of participants evaluable for DLT at each dose level. In general, the cohort size for the dose escalation phase will be 2 to 4 participants. For the dose level that is estimated to be the MTD, at least 6 (approximately 6-12) participants will be treated. Approximately 30 participants will be enrolled in Part 1A. Approximately 6-12 participants will be enrolled in each of the 2 respective combinations dose finding of the study (Part 1B and Part 1C).

### Part 2 Dose Expansion

Approximately 30 participants (approximately 40 participants in Part 2 Arm B) will be enrolled in each of the cohorts in the expansion phase of the study. Six cohorts of various tumor types, with either single agent or combination therapies, will be enrolled. The sample size is not based on statistical considerations. Rather it is based on clinical consideration that the stated sample size will provide sufficient evidence of preliminary efficacy of PF-07104091 as single agent, the combination of PF-07104091 with palbociclib and fulvestrant, or the combination of PF-07104091 with palbociclib and letrozole. A Bayesian approach will be used to estimate the ORR in the study indications. Assuming a noninformative prior (i.e., Jeffrey's prior) if 8 out of 30 participants have tumor response for example, this would translate into a posterior probability (Beta Binomial) of 82.2% that the true response is not inferior to 20%.

### Starting Dose:

The starting dose is 75 mg BID as a single agent for dose escalation. For this dose the prior risk of overdosing is satisfies the EWOC criterion. The starting dose of PF-07104091 in Part 1B and 1C will be one dose level lower than the single agent PF-07104091 MTD or an intermediate dose between MTD and the next lower dose of MTD, as guided by the BLRM and EWOC criteria, based on potential overlapping toxicity considerations and DDI assessment. The dose escalation will continue until the stopping criteria are met.

### Stopping Criteria

The dose escalation will be stopped when the following criteria are met: At least 6 participants have been treated at the recommended MTD; the dose d satisfies one of the following conditions: the probability of target toxicity at dose d exceeds 50%, and a minimum of 15 participants have been treated in the trial.

### Single Dose and Steady State PF-07104091 Pharmacokinetic Analysis

Plasma PF-07104091 concentrations will be summarized descriptively (n, mean, standard deviation, coefficient of variation, median, minimum, maximum, geometric mean and its associated coefficient of variation) by dose, cycle, day and nominal time. Individual participant plasma PF-07104091 concentration-time data within a dose interval (with exception of the food effect substudy) after Cycle 1 Day 1 and Cycle 1 Day 15 will be analyzed using noncompartmental methods to determine single and multiple dose PK parameters. For participants in the food effect substudy, individual plasma PF-07104091 concentration-time data after Cycle 1 Day 15 and Cycle 1 Day 16 will be analyzed using noncompartmental methods to determine multiple dose PK parameters. For Part 1 of the study, single dose PK parameters to be estimated will include the Cₘₐₓ, Tₘₐₓ, and AUCₗₐₛₜ, and if data permit, t_{1/2}, CL/F, and Vz/F. Multiple dose PK parameters to be estimated will include C_{max,ss}, T_{max,ss}, AUC_{T,ss}, C_{min,ss}, CL/F_{,ss}, and if data permit, V/Fₛₛ, t_{1/2}, and R_{ac}. For Part 2 of the study (with exception of the food effect substudy), single dose PK parameters to be estimated will include Cₘₐₓ, Tₘₐₓ and AUCₗₐₛₜ. Multiple dose PK parameters to be estimated will include C_{max,ss}, T_{max,ss}, AUCₗₐₛₜ, C_{min,ss}, and R_{ac}. The single dose and steady state PK parameters will be summarized descriptively (n, mean, standard deviation, coefficient of variation, median, minimum, maximum, geometric mean and its associated coefficient of variation) by dose level, cycle and day. For participants in the food effect substudy, multiple dose PK parameters to be estimated will include C_{max,ss}, AUC_{T,ss}, T_{max,ss}, C_{min,ss}, CL/Fₛₛ, and if data permit, V/Fₛₛ and t_{1/2}.

Dose normalized AUCₗₐₛₜ (AUC_{T} at steady state) and Cₘₐₓ will be plotted against dose (using a logarithmic scale) by cycle and day. These plots will include individual participant values and the geometric means for each dose.

Urine drug concentrations will be summarized by descriptive statistics (n, mean, SD, CV, median, minimum, maximum, geometric mean and its associated CV).

### Effect of Food on PF-07104091 Pharmacokinetics

The effect of food will be assessed based on AUC_{T} and Cₘₐₓ by determining the ratios (fed/fast) of geometric means of these PK parameters and the 90% confidence intervals for the ratios.

### Example 6—Pharmacokinetic Effects in Humans

An ongoing Phase 1/2a, open-label, multicenter, multiple dose study is being conducted to determine the safety, tolerability, PK, PD, and preliminary anti-tumor activity of PF-07104091 as a single agent and in combination with endocrine therapy. PF-07104091 is administered orally twice daily (BID) during 28-day cycles.

### Pharmacokinetics and Product Metabolism in Humans

Preliminary concentration-time data of PF-07104091 following single and multiple oral administration at 75 mg BID to 500 mg BID as monotherapy or in combination with endocrine therapy alone or in combination with palbociclib and endocrine therapy were available from 44 participants with advanced solid tumors and HR-positive HER2-negative advanced or metastatic breast cancer. The preliminary concentration-time data were analyzed by noncompartmental approach using actual PK sampling times.

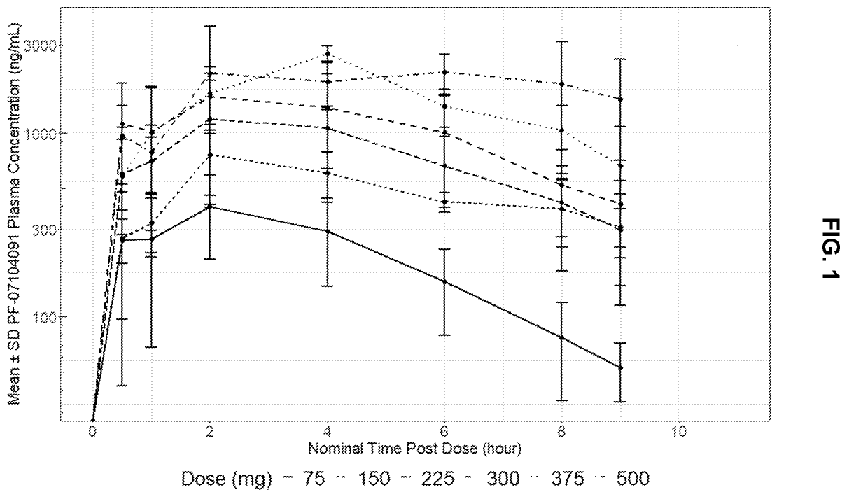

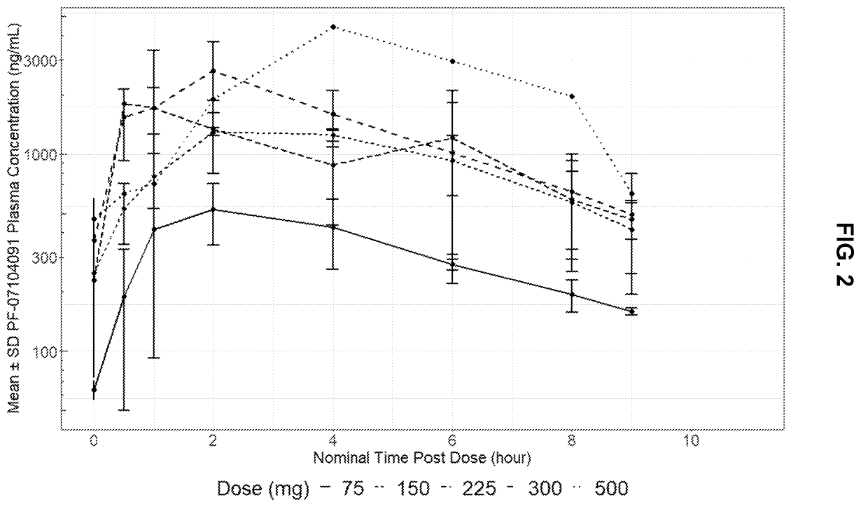

Mean±standard error (SE) PF-07104091 plasma concentration-time profiles on Cycle 1 Day 1 following a single oral dose of PF-07104091 and on Cycle 1 Day 15 following multiple oral doses of PF-07104091 are presented in FIG. 1 and FIG. 2 (semi-logarithmic scales), respectively.

Preliminary PK parameters on Cycle 1 Day 1 following a single oral dose of PF-07104091 are summarized in Table 7. Preliminary PK parameters on Cycle 1 Day 15 following repeated BID oral administration are summarized in Table 8.

TABLE 7
Preliminary Single Dose Pharmacokinetic Parameters of PF-07104091
on Cycle 1 Day 1 Following Oral Administration

| | | Cₘₐₓ | | AUCₗₐₛₜ | |
|---|---|---|---|---|---|
| Dose | N | (ng/mL) | Tₘₐₓ (h) | (ng*h/mL)^{a} | t_{1/2} (h)^{b} |
| 75 mg BID | 3 | 362 (63) | 2 (1.8-2.2) | 1754 (67) | 2.4 ± 0.7 |
| 150 mg BID | 3 | 739 (42) | 2 (2.1-7.8) | 4477 (24) | 2.8 |
| 225 mg BID | 11 | 1392 (47) | 3.9 (0.5-8) | 6634 (37) | 2.2 ± 0.7 |
| 300 mg BID | 11 | 1823 (39) | 2 (0.9-4) | 9108 (43) | 2.0 ± 0.6 |
| 375 mg BID | 2 | 2520, | 4.0, 4.0 | 11627, | 1.5 |
| | | 2890 | | 14424 | |
| 500 mg BID | 5 | 2980 (41) | 5.3 (2-8) | 15216 (26) | ND |
| 75 mg BID | 6 | 694 (36) | 1.6 (0.5-4.1) | 2760 (21) | 1.9 ± 0.2 |
| (1B) | | | | | |
| 75 mg BID | 3 | 648 (21) | 0.5 (0.5-0.5) | 2464 (10) | 1.6 ± 0.5 |
| (1C) | | | | | |

^{a}AUCₗₐₛₜ represents AUC from 0 to the last measured plasma concentration.
^{b}Estimates were based on patient data with evaluable t_{1/2} (n = 3, 1, 8, 9, 1, 5, and 3 patients for cohorts 75, 150, 225, 300, 375 mg BID monotherapy, and 75 mg BID Part 1B and 1C, respectively).

Tₘₐₓ is median (range), t_{1/2} is arithmetic mean±SD (standard deviation), and all other parameters are geometric mean coefficient of variation (CV %) for cohorts where n≥3. Individual values are listed when the number of participants with available parameters is less than 3.

TABLE 8
Preliminary Multiple Dose Pharmacokinetic Parameters
of PF-07104091 on Cycle 1 Day 15 Following
Repeated BID Oral Administration

| | | | | AUCₗₐₛₜ | | |
|---|---|---|---|---|---|---|
| | | Cₘₐₓ | Tₘₐₓ | (ng*h/ | t_{1/2} | |
| Dose | N | (ng/mL) | (h) | mL)^{a} | (h)^{b} | R_{ac} |
| 75 mg | 2 | 398, | 1.1, 2 | 2321, | ND | 1.4, 2.7 |
| BID | | 714 | | 3583 | | |
| 150 mg | 3 | 1320 | 2.2 | 8016 | 1.9 | 1.8 ± 0.2 |
| BID | | (3) | (1.8-3.9) | (11) | | |
| 225 mg | 9 | 2348 | 2 | 10705 | 2.6 ± 0.9 | 1.8 ± 0.7 |
| BID | | (33) | (0.5-6.2) | (24) | | |
| 300 mg | 7 | 2574 | 2.1 | 12469 | 2.8 ± 0.2 | 1.9 ± 1.1 |
| BID | | (36) | (0.5-4.7) | (28) | | |
| 500 mg | 1 | 4420 | 4 | 21513 | ND | 1.1 |
| BID | | | | | | |
| 75 mg | 4 | 698 | 1.2 | 3085 | 3.6 ± 0.9 | 1.2 ± 0.1 |
| BID | | (14) | (0.5-2.1) | (16) | | |
| (1B) | | | | | | |

^{a}AUCₗₐₛₜ represents AUC from 0 to the last measured plasma concentration.
^{b}Estimates were based on patient data with evaluable t_{1/2} (n = 1, 6, 5, and 3 patients for cohorts 150, 225, 300 mg BID monotherapy, and 75 mg BID Part 1B, respectively).
Tₘₐₓ is median (range), t_{1/2} and R_{ac} are arithmetic mean ± SD, and all other parameters are geometric mean (CV %) for cohorts where n ≥3. Individual values are listed when the number of participants with available parameters is less than 3.
ND = not determined

PF-07104091 was rapidly absorbed following oral administration at doses up to 300 mg BID, with a median Tₘₐₓ of 0.5 to 4 hours. Single dose and steady state Cₘₐₓ and AUCₗₐₛₜ demonstrate dose dependent increases across the dose range of 75 mg BID to 500 mg BID. The average half-life was approximately 2 hours, and there was moderate accumulation following repeated BID administration. PF-07104091 PK were similar following 75 mg BID dosing as monotherapy and in combination with palbociclib and endocrine therapy (Part 1B/1C).

### Safety Profile

In the study, 78 patients have been dosed with PF-07104091 doses ranging from 75 mg to 500 mg BID (Table 9).

TABLE 9
Phase 1/2a Dose Levels

Study Part 1A:
Cohort 1: 75 mg BID PF-07104091 single agent continuous dosing (n = 3)
Cohort 2: 150 mg BID PF-07104091 single agent continuous dosing (n = 3)
Cohort 3: 300 mg BID PF-07104091 single agent continuous dosing (n = 11)
Cohort 4: 500 mg BID PF-07104091 single agent continuous dosing (n = 5)
Cohort 5: 375 mg BID PF-07104091 single agent continuous dosing (n = 2)
Cohort 7: 225 mg BID PF-07104091 single agent continuous dosing (n = 4)
Study Part 1B:
225 mg BID PF-07104091 in combination with Fulvestrant (n = 6)
75 mg BID PF-07104091 in combination with Palbociclib 125 mg + Fulvestrant (n = 8)
150 mg BID PF-07104091 in combination with Palbociclib 100 mg + Fulvestrant (n = 10)
75 mg BID (3/1) PF-07104091 in combination with Palbociclib 125 mg + Fulvestrant (n = 3)
Study Part 1C:
75 mg BID PF-07104091 in combination with Palbociclib 125 mg + Letrozole (n = 4)
Study Part 2B:
300 mg BID PF-07104091 single agent continuous dosing (n = 12)

Of the 78 patients dosed with PF-07104091 treatment-related treatment emergent adverse event (TEAE) data is available for 77 patients (data for 2 out of 4 patients in the PF-07104091 225 mg BID group was not included). As of the data cutoff date, the following safety findings were reported: 15 DLTs; 21 SAEs, and no deaths, related to PF-07104091. 4 patients discontinued study treatment due to AEs. As of the data cutoff date, of the 80 patients that have been dosed in the study, TEAE data is available for 77 patients: A total of 70 (90.9%) out of 77 patients dosed with PF-07104091 experienced at least one all-causality TEAE. The most frequently reported (≥20%) all-causality TEAEs for PF-07104091 across all doses were nausea (68.8%), fatigue (41.6%), diarrhoea, and vomiting (37.7% each), neutrophil count decreased (32.5%), anaemia (31.2%), white blood cell count decreased (23.4%), and decreased appetite (20.8%). Majority of all-causality TEAEs reported were Grade 3 or lower (49.4%). The most frequently reported (≥5%) Grade 3 or higher all-causality TEAEs for PF-07104091 across all doses were neutrophil count decreased (23.4%), white blood cell count decreased (14.3%), fatigue (10.4%), nausea (7.8%), anaemia (6.5%), and hypokalaemia (5.2%). Four Grade 4 all-causality TEAE were reported during the study and they are as follows: neutrophil count decreased at the 500 mg BID dose (Part 1A) and 75 mg BID (3/1) (Part 1B) one event each, platelet count decreased at the 150 mg BID dose (Part 1B), and neutropenia at the 75 mg BID dose (Part 1C). One Grade 5 all-causality TEAEs of Dyspnoea at the 225 mg BID (Part A) dose was reported during the study.

### Demographics

Table 10 shows the demographics for the 78 patients enrolled in Study 04161001 as of the data cut-off date. Majority of the patients were females with the median age range between 32 to 80 years and includes patients with advanced solid tumors and HR-positive HER2-negative advanced or mBC.

TABLE 10
Study C4161001: Demographic Characteristics Categorized by PF-07104091 Treatment Group

| | PF-07104091 - Part 1A | | | | | | | | | | | | | |
|---|---|---|---|---|---|---|---|---|---|---|---|---|---|---|
| | 75 mg | | 150 mg | | 225 mg | | 300 mg | | 375 mg | | 500 mg | | | |
| | BID | | BID | | BID | | BID | | BID | | BID | | Total | |
| Characteristic | N = 3 | | N = 3 | | N = 11 | | N = 11 | | N = 2 | | N = 5 | | N = 35 | |
| Age (Years): | | | | | | | | | | | | | | |
| <18 | 0 | | 0 | | 0 | | | | | | | | | |
| 18-44 | 0 | | 0 | | 1 | (9.1%) | | | 1 | (50.0%) | 0 | | 2 | (5.7%) |
| 45-64 | 2 | (66.7%) | 1 | (33.3%) | 7 | (63.6%) | 6 | (54.5%) | 0 | | 3 | (60.0%) | 19 | (54.3%) |
| ≥65 | 1 | (33.3%) | 2 | (66.7%) | 3 | (27.3%) | 5 | (45.5%) | 1 | (50.0%) | 2 | (40.0%) | 14 | (40.0%) |
| n | 3 | | 3 | | 11 | | 11 | | 2 | | 5 | | 35 | |
| Mean (SD) | 62.7 | (7.23) | 64.7 | (2.52) | 58.2 | (11.62) | 63.0 | (6.32) | 51.0 | (26.87) | 63.0 | (4.42) | 60.9 | (9.50) |
| Median (range) | 59.0 | (58, 71) | 65.0 | (62, 67) | 59.0 | (41, 80) | 64.0 | (53, 73) | 51.0 | (32, 70) | 62.0 | (59, 69) | 62.0 | (32, 80) |
| Gender, n (%) | | | | | | | | | | | | | | |
| Male | 0 | | 0 | | 0 | | 1 | (9.1%) | 0 | | 0 | | 1 | (2.9%) |
| Female | 3 | (100.0%) | 3 | (100.0%) | 11 | (100.0%) | 10 | (90.9%) | 2 | (100.0%) | 5 | (100.0%) | 34 | (97.1%) |
| Race: | | | | | | | | | | | | | | |
| White | 3 | (100.0%) | 3 | (100.0%) | 7 | (63.6%) | 10 | (90.9%) | 2 | (100.0%) | 5 | (100.0%) | 30 | (85.7%) |
| Black or African | 0 | | 0 | | 0 | | 0 | | 0 | | 0 | | 0 | |
| American | | | | | | | | | | | | | | |
| Asian | 0 | | 0 | | 0 | | 1 | (9.1%) | 0 | | 0 | | 1 | (2.9%) |
| Not reported | 0 | | 0 | | 4 | (36.4%) | 0 | | 0 | | 0 | | 4 | (11.4%) |
| Tumor Type | | | | | | | | | | | | | | |
| Breast Cancer | 3 | (100.0) | 2 | (66.7) | 10 | (90.9) | 8 | (72.7) | 2 | (100.0) | 4 | (80.0) | 29 | (82.9) |
| Epithelial | 0 | | 1 | (33.3) | 1 | (9.1) | 1 | (9.1) | 0 | | 1 | (20.0) | 4 | (11.4) |
| Ovarian Cancer | | | | | | | | | | | | | | |
| Non-small Cell | 0 | | 0 | | 0 | | 1 | (9.1) | 0 | | 0 | | 1 | (2.9%) |
| Lung Cancer | | | | | | | | | | | | | | |
| Triple Negative | 0 | | 0 | | 0 | | 1 | (9.1) | 0 | | 0 | | 1 | (2.9%) |
| Breast Cancer | | | | | | | | | | | | | | |
| Baseline ECOG | | | | | | | | | | | | | | |
| Status | | | | | | | | | | | | | | |
| 0 | 0 | | 0 | | 1 | (9.1%) | 5 | (45.5%) | 0 | | 1 | (20.0%) | 7 | (20.0%) |
| 1 | 3 | (100.0%) | 3 | (100.0%) | 9 | (81.8%) | 6 | (54.5%) | 2 | (100.0%) | 4 | (80.0%) | 27 | (77.1%) |

| | | | | | | | | | |
|---|---|---|---|---|---|---|---|---|---|
| Not Reported | 0 | 0 | 1 | (9.1%) | 0 | 0 | 0 | 1 | (2.9%) |

TABLE 10
Study C4161001: Demographic Characteristics Categorized by PF-07104091 Treatment Group (Cont.)

| | PF-07104091 | | | | | | | | | |
|---|---|---|---|---|---|---|---|---|---|---|
| | Part 1B | | | | | | | | | |
| | | | | | | | 75 mg | | | |
| | 75 mg | | | | 150 mg | | BID (3/1) + | | | |
| | BID + Palbo | | 225 mg | | BID + Palbo | | Palbo | | | |
| | 125 mg + | | BID + | | 100 mg + | | 125 mg + | | | |
| | Fulvestrant | | Fulvestrant | | Fulvestrant | | Fulvestrant | | Total | |
| Characteristic | N = 8 | | N = 6 | | N = 10 | | N = 3 | | N = 27 | |
| Age (Years): | | | | | | | | | | |
| <18 | 0 | | 0 | | 0 | | 0 | | 0 | |
| 18-44 | 0 | | 1 | (16.7%) | 0 | | 0 | | 1 | (3.7%) |
| 45-64 | 8 | (100.0%) | 3 | (50.0%) | 6 | (60.0%) | 2 | (66.7%) | 19 | (70.4%) |
| ≥65 | 0 | | 2 | (33.3%) | 4 | (40.0%) | 1 | (33.3%) | 7 | (25.9%) |
| n | 8 | | 6 | | 10 | | 3 | | 27 | |
| Mean (SD) | 54.5 | (4.04) | 60.2 | (13.11) | 61.7 | (8.55) | 63.0 | (10.54) | 59.4 | (9.07) |
| Median (range) | 54.5 | (50, 63) | 61.5 | (41, 77) | 63.0 | (49, 78) | 62.0 | (53, 74) | 59.0 | (41, 78) |
| Gender, n (%) | | | | | | | | | | |
| Male | 0 | | 0 | | 0 | | 0 | | 0 | |
| Female | 8 | (100.0%) | 6 | (100.0%) | 10 | (100.0%) | 3 | (100.0%) | 27 | (100.0%) |
| Race: | | | | | | | | | | |
| White | 7 | (87.5%) | 6 | (100.0%) | 9 | (90.0%) | 3 | (100.0%) | 25 | (92.6%) |
| Black or | 0 | | 0 | | 0 | | 0 | | 0 | |
| African | | | | | | | | | | |
| American | | | | | | | | | | |
| Asian | 1 | (12.5%) | 0 | | 1 | (10.0%) | 0 | | 2 | (7.4%) |
| Tumor Type | | | | | | | | | | |
| ER+ PR + | 3 | (37.5) | 6 | (100.0) | 9 | (90.0) | 3 | (100.0) | 21 | (77.8) |
| HER2-Breast | | | | | | | | | | |
| Cancer | | | | | | | | | | |
| Epithelial | 5 | (62.5) | 0 | | 0 | | 0 | | 5 | (18.5) |
| Ovarian Cancer | | | | | | | | | | |
| ER+ PR − | 0 | | 0 | | 1 | (10.0) | 0 | | 1 | (3.7) |
| HER2-Breast | | | | | | | | | | |
| Cancer | | | | | | | | | | |
| Epithelial | 0 | | 0 | | 0 | | 0 | | 0 | |
| Ovarian Cancer | | | | | | | | | | |
| Fallopian | 0 | | 0 | | 0 | | 0 | | 0 | |
| Tube Cancer | | | | | | | | | | |
| Small Cell | 0 | | 0 | | 0 | | 0 | | 0 | |
| Lung Cancer | | | | | | | | | | |
| Baseline | | | | | | | | | | |
| ECOG Status | | | | | | | | | | |
| 0 | 4 | (50.0%) | 1 | (16.7%) | 5 | (50.0%) | 1 | (33.3%) | 11 | (40.7%) |
| 1 | 3 | (37.5%) | 5 | (83.3%) | 5 | (50.0%) | 2 | (66.7%) | 15 | (55.6%) |

| | | | | | | | |
|---|---|---|---|---|---|---|---|
| Not Reported | 1 | (12.5%) | 0 | 0 | 0 | 1 | (3.7%) |

| | PF-07104091 | | | | | | | |
|---|---|---|---|---|---|---|---|---|
| | Part 1C | | | | | | | |
| | 75 mg | | | | | | | |
| | BID + | | | | | | | |
| | Palbo | | | | Part 2B | | | |
| | 125 mg + | | | | 300 mg | | | |
| | Letrozole | | Total | | BID | | Total | |
| Characteristic | N = 4 | | N = 4 | | N = 12 | | N = 12 | |
| Age (Years): | | | | | | | | |
| <18 | 0 | | 0 | | 0 | | 0 | |
| 18-44 | 0 | | 0 | | 0 | | 0 | |
| 45-64 | 4 | (100.0%) | 4 | (100.0%) | 3 | (25.0%) | 3 | (25.0%) |
| ≥65 | 0 | | 0 | | 9 | (75.0%) | 9 | (75.0%) |
| n | 4 | | 4 | | 12 | | 12 | |
| Mean (SD) | 58.5 | (4.80) | 58.5 | (4.80) | 65.4 | (9.39) | 65.4 | (9.39) |
| Median (range) | 59.5 | (52, 63) | 59.5 | (52, 63) | 68.0 | (46, 80) | 68.0 | (46, 80) |
| Gender, n (%) | | | | | | | | |
| Male | 0 | | 0 | | 1 | (8.3%) | 1 | (8.3%) |
| Female | 4 | (100.0%) | 4 | (100.0%) | 11 | (91.7%) | 11 | (91.7%) |
| Race: | | | | | | | | |
| White | 4 | (100.0%) | 4 | (100.0%) | 10 | (83.3%) | 10 | (83.3%) |
| Black or | 0 | | 0 | | 0 | | 0 | |
| African | | | | | | | | |
| American | | | | | | | | |
| Asian | 0 | | 0 | | 2 | (16.7%) | 2 | (16.7%) |
| Tumor Type | | | | | | | | |
| ER+ PR + | 0 | | 0 | | 0 | | 0 | |
| HER2-Breast | | | | | | | | |
| Cancer | | | | | | | | |
| Epithelial | 0 | | 0 | | 0 | | 0 | |
| Ovarian Cancer | | | | | | | | |
| ER+ PR − | 4 | (100.0) | 4 | (100.0) | 0 | | 0 | |
| HER2-Breast | | | | | | | | |
| Cancer | | | | | | | | |
| Epithelial | 0 | | 0 | | 10 | (83.3) | 10 | (83.3) |
| Ovarian Cancer | | | | | | | | |
| Fallopian | 0 | | 0 | | 1 | (8.3) | 1 | (8.3) |
| Tube Cancer | | | | | | | | |
| Small Cell | 0 | | 0 | | 1 | (8.3) | 1 | (8.3) |
| Lung Cancer | | | | | | | | |
| Baseline | | | | | | | | |
| ECOG Status | | | | | | | | |
| 0 | 3 | (75.0%) | 3 | (75.0%) | 6 | (50.0%) | 6 | (50.0%) |
| 1 | 1 | (25.0%) | 1 | (25.0%) | 6 | (50.0%) | 6 | (50.0%) |

| | | | | |
|---|---|---|---|---|
| Not Reported | 0 | 0 | 0 | 0 |

What is claimed:

1. A method of treating cancer in a subject in need thereof comprising administering to the subject an effective amount of PF-07104091, wherein the effective amount is from about 75 mg to about 500 mg twice per day (BID).

2. The method of claim 1, wherein the effective amount is from about 150 mg to about 300 mg BID.

3. The method of claim 1 or 2, wherein the effective amount is about 150 mg BID, about 225 mg BID, or about 300 mg BID.

4. A method of treating cancer in a subject in need thereof comprising administering to the subject an effective amount of PF-07104091, wherein the effective amount is from about 150 mg to about 1000 mg per day.

5. The method of claim 4, wherein the effective amount is from about 300 mg to about 600 mg per day.

6. The method of claim 4 or 5, wherein the effective amount is about 300 mg per day, about 450 mg per day, or about 600 mg per day.

7. The method of any one of claims 1 to 6, wherein PF-07104091 is administered continuously on a 28 day cycle.

8. The method of any one of claims 1 to 7, wherein PF-07104091 is administered as a tablet or capsule dosage form.

9. A method of treating cancer in a subject in need thereof comprising administering to the subject an amount of PF-07104091 and an amount of an endocrine therapy agent, wherein the amount of PF-07104091 is from about 75 mg to about 500 mg BID, and the amounts of PF-07104091 and the endocrine therapy agent are together effective to treat cancer.

10. The method of claim 9, wherein the endocrine therapy agent is an aromatase inhibitor, a selective estrogen receptor degrader (SERD), or a selective estrogen receptor modulator (SERM).

11. The method of claim 10, wherein the endocrine therapy agent is selected from the group consisting of letrozole, anastrozole, exemestane, fulvestrant, elacestrant, amcenestrant, giredestrant, RG6171, camizestrant, AZD9496, rintodestrant, ZN-c5, LSZ102, D-0502, LY3484356, SHR9549, tamoxifen, raloxifene, toremifene, lasofoxifene, bazedoxifene and afimoxifene.

12. The method of any one of claims 9 to 11, wherein the endocrine therapy agent is letrozole or fulvestrant.

13. The method of any one of claims 9 to 12, wherein the subject is administered the endocrine therapy agent and PF-07104091 sequentially, concurrently or simultaneously.

14. The method of any one of claims 1 or 13, wherein the subject has been previously treated with chemotherapy, radiotherapy, and/or surgical resection.

15. The method of any one of claims 1 or 13, wherein the subject has been previously treated with a CDK4/6 inhibitor.

16. The method of any one of claims 1 or 13, wherein the subject has been previously treated with an endocrine therapy agent.

17. The method of any one of claims 1 or 16, wherein the subject is a human.

18. The method of any one of claims 1 to 17, wherein the cancer is breast cancer, prostate cancer, lung cancer, liver cancer, kidney cancer, bladder cancer, ovarian cancer, peritoneal cancer, fallopian tube cancer, cervical cancer, uterine cancer, pancreatic cancer, stomach cancer, colorectal cancer, esophageal cancer, head and neck cancer, testicular cancer, adrenal cancer, skin cancer, brain cancer, sarcoma, and lymphoma.

19. The method of any one of claims 1 to 18, wherein the cancer is breast cancer selected from hormone receptor positive (HR+), human epidermal growth factor receptor 2 negative (HER2−) breast cancer and triple negative breast cancer (TNBC).

20. The method of any one of claims 1 to 18, wherein the cancer is lung cancer selected from small cell lung cancer (SCLC) and non-small cell lung cancer (NSCLC).

21. A method of treating cancer in a subject in need thereof comprising administering to the subject an effective amount of PF-07104091 monohydrate, wherein the effective amount is from about 75 mg to about 500 mg twice per day (BID).

22. A method of treating cancer in a subject in need thereof comprising administering to the subject an effective amount of PF-07104091 monohydrate, wherein the effective amount is from about 150 mg to about 1000 mg per day.

23. The method of any one of claims 1 to 22, further comprising administering to the subject an effective amount of a CDK4/6 inhibitor.

24. The method of claim 23, wherein the CDK4/6 inhibitor is palbociclib, and wherein the effective amount of palbociclib is about 75 mg once a day (QD), about 100 mg QD, or about 125 mg QD.

25. The method of claim 24, wherein palbociclib is administered on a 28 day cycle with 21 days on treatment followed by 7 days off treatment with palbociclib.

26. The method of claim 23, wherein the CDK4/6 inhibitor is abemaciclib or ribociclib.
